(19) United States
(12) Patent Application Publication
(10) Pub. No.: US 2014/0135370 A1
(43) Pub. Date: May 15, 2014

(54) TREATING CANCER WITH AN HSP90 INHIBITORY COMPOUND

(71) Applicant: Vojo Vukovic, Winchester, MA (US)

(72) Inventor: Vojo Vukovic, Winchester, MA (US)

(73) Assignee: SYNTA PHARMACEUTICALS CORP., Lexington, MA (US)

(21) Appl. No.: 14/075,762

(22) Filed: Nov. 8, 2013

Related U.S. Application Data
(63) Continuation of application No. PCT/US2012/037564, filed on May 11, 2012.
(60) Provisional application No. 61/583,773, filed on Jan. 6, 2012.
(60) Provisional application No. 61/578,459, filed on Dec. 21, 2011.
(60) Provisional application No. 61/567,942, filed on Dec. 7, 2011.
(60) Provisional application No. 61/565,126, filed on Nov. 30, 2011.
(60) Provisional application No. 61/538,400, filed on Sep. 23, 2011.
(60) Provisional application No. 61/504,417, filed on Jul. 5, 2011.
(60) Provisional application No. 61/498,966, filed on Jun. 20, 2011.
(60) Provisional application No. 61/493,063, filed on Jun. 3, 2011.
(60) Provisional application No. 61/489,867, filed on May 25, 2011.
(60) Provisional application No. 61/484,988, filed on May 11, 2011.
(60) Provisional application No. 61/484,992, filed on May 11, 2011.

(51) Int. Cl. A61K 31/4196; A61K 31/337

(52) U.S. Cl. CPC A61K 31/4196; A61K 31/337
USPC 514/384

(57) ABSTRACT

Methods of treating cancer with a compound of formula (I) are disclosed. Also provided are methods of treating a cancer with a KRAS mutation or an ALK+ cancer with a compound of formula (I). Further provided are methods of treating KRAS− mutated or ALK+ NSCLC with a compound of formula (I):

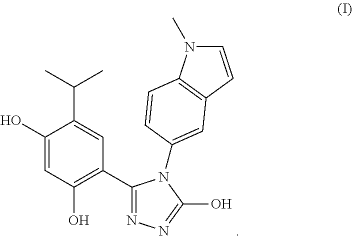 [SMILES: CC(C)c1cc(-c2nnc(O)n2-c2ccc3c(ccn3C)c2)c(O)cc1O]

### CROSS-REFERENCE TO RELATED PATENTS

This application claims the benefit of priority to U.S. Provisional Patent Application Nos. 61/484,988 and 61/484,992, filed on May 11, 2011; 61/489,867, filed on May 25, 2011; 61/493,063; filed on Jun. 3, 2011; 61/498,966, filed on Jun. 20, 2011; 61/504,417, filed on Jul. 5, 2011; 61/538,400, filed on Sep. 23, 2011; 61/565,126, filed on Nov. 30, 2011; 61/567,942, filed on Dec. 7, 2011; 61/578,459, filed on Dec. 21, 2011; and 61/583,773, filed on Jan. 6, 2012. The contents of each of these applications are incorporated herein by reference in their entireties.

### FIELD OF THE INVENTION

This invention relates to the use of Hsp90 inhibitors in treating humans with certain specific types of cancer. Regimens disclosed herein demonstrate potency against certain specific types of cancer, while showing minimal side effects.

### BACKGROUND OF THE INVENTION

Although tremendous advances have been made in elucidating the genomic abnormalities that cause cancer, currently available chemotherapy remains unsatisfactory, and the prognosis for the majority of patients diagnosed with cancer remains poor. Many chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called “client” proteins), facilitate their proper folding and repair, and aid in the refolding of misfolded client proteins. There are several known families of HSPs, each having its own set of client proteins. The Hsp90 family is one of the most abundant HSP families, accounting for about 1-2% of proteins in a cell that is not under stress, increasing to about 4-6% in a cell under stress Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

Triazolone Hsp90 inhibitors are demonstrated herein to be particularly effective in specific dosing regimens for treating humans with cancer. It is also demonstrated herein that those Hsp90 inhibitors are particularly effective in treating certain specific types of cancer, including cancers having a mutation in one or more of KRAS, epidermal growth factor receptor (EGFR), or anaplastic lymphoma kinase (ALK). The particular dosing regimens disclosed herein demonstrate potency against certain specific types of cancer, while showing minimal side effects.

In one embodiment, an Hsp90 inhibitor of formula (I) (ganetespib), or a pharmaceutically acceptable salt or tautomer thereof, is useful for the treatment of cancer:

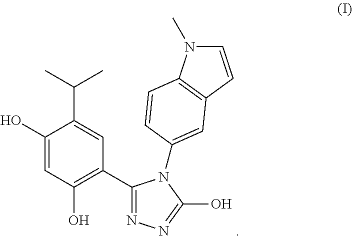 [SMILES: CC(C)c1cc(-c2nnc(O)n2-c2ccc3c(ccn3C)c2)c(O)cc1O]

In one embodiment, the treatment method includes administering to a subject an effective amount of the compound of formula (I) from about 2 mg/m² to about 260 mg/m². In one embodiment, the compound of formula (I) is administered once weekly.

In one embodiment, the compound of formula (I) is administered twice-weekly. In one embodiment, the compound of formula (I) is administered for about 3 weeks. In another embodiment, the administration for 3 weeks is repeated after about 7 days dose-free. In one embodiment, the administration after 7 days dose-free is repeated at two or more times. In one embodiment, the compound of formula (I) is administered by intravenous infusion, such as peripheral intravenous infusion. In one embodiment, the compound of formula (I) is infused over 60 minutes.

In one embodiment, the method is used for treating a subject with a non-small cell lung cancer (“NSCLC”). In one embodiment, the NSCLC expresses wild-type EGFR and wild-type KRAS. In one embodiment, the NSCLC has an EGFR mutation.

In one embodiment, the NSCLC has a KRAS mutation. In one embodiment, the NSCLC has an EGFR mutation or a KRAS mutation. In one embodiment, the NSCLC is an anaplastic lymphoma kinase positive (“ALK+”) NSCLC (i.e., it has an ALK mutation). In one embodiment, the NSCLC is refractory. In one embodiment, the subject was previously treated with other anticancer agents. In one embodiment, the subject was previously treated with crizotinib. In certain embodiments, the subject was treated with crizotinib and the NSCLC became resistant to the crizotinib treatment. In one embodiment, the cancer is stage IIIB or IV NSCLC.

In one embodiment, the compound of formula (I) is used for treating a subject with cancer with a KRAS mutation. In one embodiment, the treatment method includes administering to the subject with a cancer with a KRAS mutation an effective amount of the compound of formula (I), or a pharmaceutically acceptable salt or tautomer thereof.

In one embodiment, the compound of formula (I) is used for treating a subject with a cancer with a KRAS mutation in combination with one or more additional anticancer agents. In one embodiment, the compound of formula (I) is used for treating cancer in a subject with a KRAS mutation in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

In one embodiment, the compound of formula (I) is used for treating a subject with NSCLC with a KRAS mutation. In one embodiment, the treatment method includes administering to the subject with NSCLC with a KRAS mutation an effective amount of the compound of formula (I), or a pharmaceutically acceptable salt or tautomer thereof. In one embodiment, the compound of formula (I) is used for treating a subject with NSCLC with a KRAS mutation in combination with one or more additional anticancer agents. In one embodiment, the compound of formula (I) is used for treating NSCLC in a subject with a KRAS mutation in combination with BEZ235, AZD6244, AZD8055, SN-38, gemcitabine, camptothecin, docetaxel, cisplatin, oxaliplatin, crizotinib, paclitaxel, trastuzumab, and pemetrexed.

In one embodiment, the method of treating a subject with a cancer with a KRAS mutation includes:
a) identifying a subject with a cancer with a KRAS mutation; and
b) administering to the subject an effective amount of a compound of formula (I) or a pharmaceutically acceptable salt or tautomer thereof.

In one embodiment, the compound of formula (I) is used for treating a subject with an ALK+ cancer. In one embodiment, the treatment method includes administering to the subject with an ALK+ cancer an effective amount of the compound of formula (I), or a pharmaceutically acceptable salt or tautomer thereof. In one embodiment, the compound of formula (I) is used for treating a subject with an ALK+ cancer in combination with one or more additional anticancer agents. In one embodiment, the compound of formula (I) is used for treating an ALK+ cancer in a subject in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

In one embodiment, the compound of formula (I) is used for treating a subject with ALK+ NSCLC. In one embodiment, the treatment method includes administering to the subject with ALK+ NSCLC an effective amount of the compound of formula (I), or a pharmaceutically acceptable salt or tautomer thereof. In one embodiment, the compound of formula (I) is used for treating a subject with ALK+ NSCLC in combination with one or more additional anticancer agents. In one embodiment, the compound of formula (I) is used for treating ALK+ NSCLC in a subject in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

In one embodiment, the method of treating cancer in a subject with an ALK mutation includes:
a) identifying a subject with cancer with an ALK mutation; and
b) administering to the subject an effective amount of a compound of formula (I) or a pharmaceutically acceptable salt or tautomer thereof.

In one embodiment, the method is used for treating breast cancer, gastric cancer, colorectal cancer, pancreatic cancer, ocular melanoma, prostate cancer, melanoma, gastrointestinal stromal tumors (GIST), advanced esophagogastric cancer, hepatocellular cancer, solid tumor, small cell lung cancer, head and neck cancer, or a hematological malignancy. In one embodiment, the breast cancer is triple negative breast cancer (i.e., estrogen receptor (ER) negative/progesterone receptor (PR) negative/human epidermal growth factor receptor 2 (HER2) negative), invasive ductal carcinoma, or metastatic breast cancer. In one embodiment, the breast cancer is HER2 positive and trastuzumab refractory. In one embodiment, the breast cancer is HER2 positive and the subject has been previously treated with trastuzumab.

In one embodiment, the method is used for treating ocular melanoma, pancreatic cancer, prostate cancer, solid tumor, hepatocellular cancer, colorectal cancer, or small cell lung cancer. In one embodiment, the ocular melanoma is metastatic. In one embodiment, the pancreatic cancer is metastatic. In one embodiment, the prostate cancer is metastatic hormone-resistant prostate cancer. In one embodiment, the prostate cancer is metastatic castration-resistant prostate cancer (CRPC). In one embodiment, the subject with prostate cancer was previously treated with docetaxel-based chemotherapy. In one embodiment, the cancer is a solid tumor. In one embodiment, the cancer is advanced hepatocellular cancer. In one embodiment, the colorectal cancer is refractory metastatic colorectal cancer. In one embodiment, the small cell lung cancer is relapsed or refractory.

In one embodiment, the compound of formula (I) is used for treating lung cancer in combination with an MEK inhibitor. In one embodiment, the compound of formula (I) is used for treating lung cancer in combination with an MEK inhibitor and a PI3K/mTOR inhibitor. In one embodiment, the compound of formula (I) is used for treating lung cancer in combination with a PI3K/mTOR inhibitor.

### BRIEF DESCRIPTION OF THE DRAWINGS

FIG. 1 shows the activity of various chemotherapeutic agents: Compound of formula (I), an mTOR/PI3K inhibitor, a MEK inhibitor, and a HER2/EGFR inhibitor, as indicated, in a 72 hr viability assay using MDA-MB-231 breast cancer cells.

FIG. 2 shows the activity of the compound of formula (I) in a 24 hr viability assay using SUM149 inflammatory breast cancer (IBC) cells.

FIG. 3 shows the activity of the compound of formula (I) in a viability assay in BT-474 breast cancer cells grown as mammospheres in Matrigel®. Figures show images of BT-474 mammospheres treated with vehicle control, 41 nM and 124 nM Compound of formula (I), and 41 nM and 124 nM Lapatinib, as indicated. The cells were treated for 72 hr and analyzed by microscopy. IC₅₀ was determined by AlamarBlue®.

FIG. 4 A shows the activity of the compound of formula (I) in a single agent viability assay Detroit562 cells, a head and neck cancer cell line, exposed to various chemotherapeutic agents: 17-AAG, Doxorubicin, Oxaliplatin, Carboplatin, and Cisplatin, as indicated, for 72 hr (left).

FIG. 4B shows the expression of various HSP90 client proteins: EGFR, Jak2, P-Stat3, Stat-3, p70 S6 kinase, Cleaved Parp, HSP90α, HSP70, P-Akt, Akt, P-Erk, Erk, Cdc2, and Gapdh (control), as indicated, as determined by western blot of cell extracts from Detroit562 cells exposed to the compound of formula (I) for 24 hr (right).

FIG. 5 shows a western blot of protein expression in cell extracts from Detroit 562 head and neck cancer cells treated with 100 nM of the compound of formula (I) 24 hours prior to receiving the DNA damaging agent bleomycin (5 μM). Protein expression of Parp, P-Chk1, P-Chk2, and Gapdh (control) was measured at the indicated time points (1, 4, and 8 hours) after bleomycin treatment. Bleomycin increased both Chk1 and Chk2 phosphorylation, which was blocked when cells were treated first with compound of formula (I).

FIG. 6 is a waterfall diagram showing the best percentage changes in size of target lesions responses according to ALK status after treatment with the compound of formula (I). The y axis represents the percentage tumor volume change from baseline. For each patient (each bar) the percentage change in measurable tumor at best response was displayed by the genotype of the patient, i.e., ALK status. A subject was considered to be ALK+ (i.e., have an ALK mutation) if a mutation in ALK was detected using any of the methods.

FIG. 7 shows a western blot of HSP90 client proteins P-HER2, P-EGFR, P-4EBP1, Cyclin D1, PARP, and GAPDH (control) in BT-474 cells after treatment with the compound of formula (I) for 16 hours.

FIG. 8 shows a graph of the average tumor volume over time in an MDA-MB-231 xenograft model in response to treatment with the compound of formula (I).

FIG. 9 is a waterfall diagram showing the best response in patients with metastatic breast cancer based on ER, PR, and HER2 marker status in a Phase II clinical trial of the compound of formula (I).

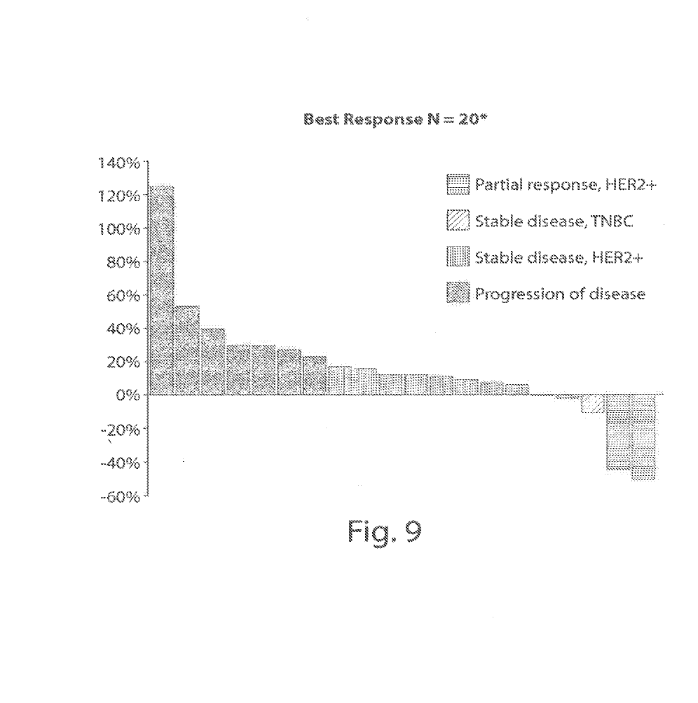

FIG. 10 shows a PET/CT scan of the lungs and bone before (left panel) and after (right panel) 19 days of treatment with the compound of formula (I) in a female patient with metastatic triple negative breast cancer. Arrows indicate the tumor mass in the lung.

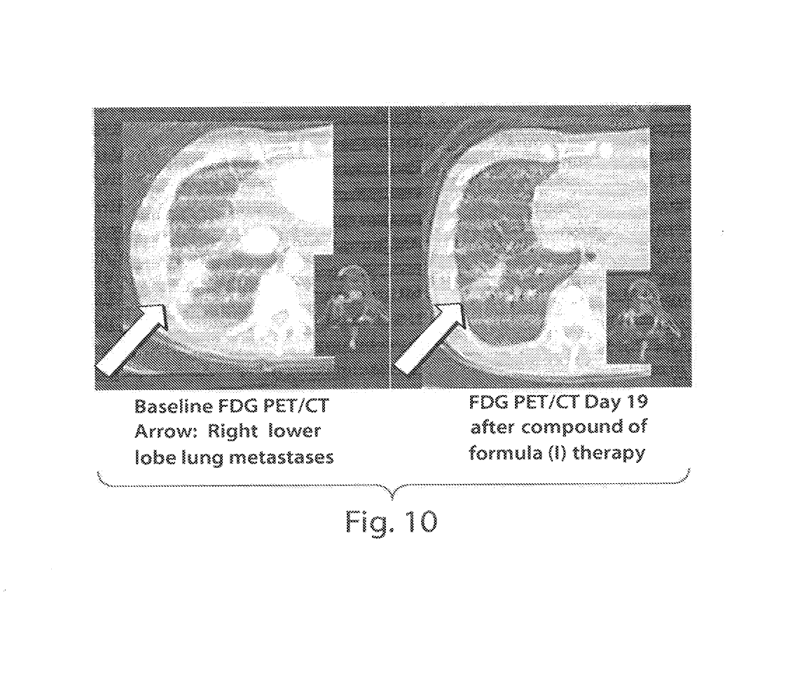

FIG. 11 shows a table of IC₅₀ values for the compound of formula (I) in NSCLC cell lines with a KRAS mutation after treatment with the compound of formula (I) for 72 hr.

FIG. 12 shows a graph of the results of treatment of various NSCLC cell lines with the compound of formula (I), camptothecin, or a combination thereof for 72 hours.

FIG. 13 shows a graph of the results of treatment of various NSCLC cell lines with NSCLC cells with the compound of formula (I), pemetrexed, or a combination thereof for 72 hours.

FIG. 14 shows a graph of the results of treatment of various NSCLC cell lines with the compound of formula (I), gemcitabine, or a combination thereof for 72 hours.

FIG. 15 shows a graph of the results of treatment of various NSCLC cell lines with the compound of formula (I), certain platins, or a combination thereof for 72 hours.

FIG. 16 shows a graph of the results of treatment of various NSCLC cell lines with the compound of formula (I), SN-38, or a combination thereof for 72 hours.

FIG. 17 shows a graph of the results of treatment of various NSCLC cell lines with the compound of formula (I), docetaxel, or a combination thereof for 72 hours.

FIG. 18 shows a graph of the results of treatment of various NSCLC cell lines with the compound of formula (I), AZD6244, or a combination thereof for 72 hours.

FIG. 19 shows a graph of the results of treatment of various NSCLC cell lines with the compound of formula (I), BEZ235, or a combination thereof for 72 hours.

FIG. 20 shows a graph of the results of treatment of mice with A549 NSCLC xenografts with the compound of formula (I), BEZ-235, or a combination thereof.

### DETAILED DESCRIPTION OF THE INVENTION

The invention, in an embodiment, provides the use of an Hsp90 inhibitor of formula (I) (ganetespib), or a pharmaceutically acceptable salt or tautomer thereof:

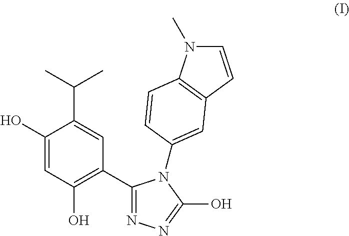 [SMILES: CC(C)c1cc(-c2nnc(O)n2-c2ccc3c(ccn3C)c2)c(O)cc1O]

for the treatment of cancer.

In an embodiment, the treatment method includes administering to a subject an effective amount of the compound of formula (I) from about 2 mg/m² to about 260 mg/m². In an embodiment, the compound of formula (I) is administered once weekly. In an embodiment, the compound of formula (I) is administered twice-weekly. In an embodiment, the compound of formula (I) is administered for about 3 weeks. In another embodiment, the administration for 3 weeks is repeated after about 7 days dose-free. In an embodiment, the administration after 7 days dose-free is repeated at two or more times. In an embodiment, the compound of formula (I) is administered by intravenous infusion, such as peripheral intravenous infusion. In an embodiment, the compound of formula (I) is infused over 60 minutes.

In an embodiment, the method is used for treating a subject with NSCLC. In an embodiment, the NSCLC expresses wild-type EGFR and wild-type KRAS. In an embodiment, the NSCLC has an EGFR mutation. In an embodiment, the NSCLC has a KRAS mutation. In an embodiment, the NSCLC has an EGFR mutation and a KRAS mutation. In an embodiment, the NSCLC is ALK+(i.e., it has an ALK mutation.) In an embodiment, the NSCLC is refractory. In an embodiment, the NSCLC was previously treated with other anticancer agents. In an embodiment, the NSCLC was previously treated with crizotinib. In an embodiment, the NSCLC was treated with and became resistant to the crizotinib treatment. In an embodiment, the cancer is stage IIIB or IV NSCLC.

In an embodiment, the compound of formula (I) is used for treating a subject with cancer with a KRAS mutation. In an embodiment, the treatment method includes administering to the subject with a cancer with a KRAS mutation an effective amount of the compound of formula (I), or a pharmaceutically acceptable salt or tautomer thereof. In an embodiment, the compound of formula (I) is used for treating a subject with a cancer with a KRAS mutation in combination with one or more additional anticancer agents. In an embodiment, the compound of formula (I) is used for treating a subject with a cancer with a KRAS mutation in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

In an embodiment, the compound of formula (I) is used for treating a subject with NSCLC with a KRAS mutation. In an embodiment, the treatment method includes administering to the subject with a NSCLC with a KRAS mutation an effective amount of the compound of formula (I), or a pharmaceutically acceptable salt or tautomer thereof. In an embodiment, the compound of formula (I) is used for treating a subject with NSCLC with a KRAS mutation in combination with one or more additional anticancer agents. In an embodiment, the compound of formula (I) is used for treating a subject with NSCLC with a KRAS mutation in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

In an embodiment, the method of treating a subject with a cancer with a KRAS mutation, includes:
a) identifying a subject with a mutation in a KRAS gene; and
b) administering to the subject an effective amount of a compound of formula (I) or a pharmaceutically acceptable salt or tautomer thereof.

In an embodiment, the method further comprises administering one or more additional anticancer drugs. In an embodiment, the one or more drugs are selected from the group consisting of BEZ235, AZD6244, AZD8055, SN-38, gemcitabine, camptothecin, docetaxel, cisplatin, oxaliplatin, crizotinib, paclitaxel, trastuzumab, and pemetrexed.

In an embodiment, the method of treating a subject with a NSCLC with a KRAS mutation includes:
a) identifying a subject with a mutation in a KRAS gene; and
b) administering to the subject an effective amount of a compound of formula (I) or a pharmaceutically acceptable salt or tautomer thereof. In an embodiment, the method further comprises administering one or more additional anticancer drugs.

In an embodiment, the one or more drugs are selected from the group consisting of BEZ235, AZD6244, AZD8055, SN-38, gemcitabine, camptothecin, docetaxel, cisplatin, oxaliplatin, crizotinib, paclitaxel, trastuzumab, and pemetrexed.

In an embodiment, the compound of formula (I) is used for treating a subject with an ALK+ cancer. In an embodiment, the treatment method includes administering to the subject with an ALK+ cancer an effective amount of the compound of formula (I), or a pharmaceutically acceptable salt or tautomer thereof. In an embodiment, the compound of formula (I) is used for treating a subject with an ALK+ cancer in combination with one or more additional anticancer agents. In an embodiment, the compound of formula (I) is used for treating an ALK+ cancer in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

In an embodiment, the compound of formula (I) is used for treating a subject with ALK+ NSCLC. In an embodiment, the treatment method includes administering to the subject with ALK+ NSCLC an effective amount of the compound of formula (I), or a pharmaceutically acceptable salt or tautomer thereof. In an embodiment, the compound of formula (I) is used for treating a subject with ALK+ NSCLC in combination with one or more additional anticancer agents. In an embodiment, the compound of formula (I) is used for treating ALK+ NSCLC in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

In an embodiment, the method of treating a subject with a cancer with an ALK mutation includes:
a) identifying a subject with a mutation in an ALK gene; and
b) administering to the subject an effective amount of a compound of formula (I) or a pharmaceutically acceptable salt or tautomer thereof. I

In an embodiment, the method further comprises administering one or more additional anticancer drugs. In an embodiment, the one or more drugs are selected from the group consisting of BEZ235, AZD6244, AZD8055, SN-38, gemcitabine, camptothecin, docetaxel, cisplatin, oxaliplatin, crizotinib, paclitaxel, trastuzumab, and pemetrexed.

In an embodiment, the method of treating a subject with a NSCLC with an ALK mutation includes:
a) identifying a subject with a mutation in an ALK gene; and
b) administering to the subject an effective amount of a compound of formula (I) or a pharmaceutically acceptable salt or tautomer thereof.

In an embodiment, the method further comprises administering one or more additional anticancer drugs. In an embodiment, the one or more drugs are selected from the group consisting of BEZ235, AZD6244, AZD8055, SN-38, gemcitabine, camptothecin, docetaxel, cisplatin, oxaliplatin, crizotinib, paclitaxel, trastuzumab, and pemetrexed.

In certain embodiments, the methods are used for treating breast cancer, gastric cancer, colorectal cancer, pancreatic cancer, ocular melanoma, prostate cancer, melanoma, gastrointestinal stromal tumors (GIST), advanced esophagogastric cancer, hepatocellular cancer, solid tumor, small cell lung cancer, head and neck cancer, or hematological malignancies. In an embodiment, the breast cancer is triple negative breast cancer, invasive ductal carcinoma, or metastatic breast cancer. In an embodiment, the breast cancer is HER2 positive and trastuzumab refractory. In an embodiment, the breast cancer is HER2 positive and has been previously treated with trastuzumab. In an embodiment, the method is for treating triple negative breast cancer in combination with an additional anticancer agent. In an embodiment, the method is for treating triple negative breast cancer, or HER2 positive cancer in combination with BEZ235, AZD6244, AZD8055, SN-38, gemcitabine, camptothecin, docetaxel, cisplatin, oxaliplatin, crizotinib, paclitaxel, trastuzumab, or pemetrexed. In an embodiment, the method is for treating triple negative breast cancer or HER2 positive cancer in combination with trastuzumab.

In certain embodiments, the methods are used for treating ocular melanoma, pancreatic cancer, prostate cancer, solid tumor, hepatocellular cancer, colorectal cancer, or small cell lung cancer. In an embodiment, the ocular melanoma is metastatic. In an embodiment, the pancreatic cancer is metastatic. In an embodiment, the prostate cancer is metastatic hormone-resistant prostate cancer. In an embodiment, the prostate cancer is metastatic castration-resistant prostate cancer (CRPC). In an embodiment, the subject with prostate cancer was previously treated with docetaxel-based chemotherapy. In an embodiment, the cancer is a solid tumor. In an embodiment, the cancer is advanced hepatocellular cancer. In an embodiment, the colorectal cancer is refractory metastatic colorectal cancer. In an embodiment, the small cell lung cancer is relapsed or refractory.

In an embodiment, the compound of formula (I) is used for treating lung cancer in combination with an MEK inhibitor. In an embodiment, the compound of formula (I) is used for treating lung cancer in combination with an MEK inhibitor and a PI3K/mTOR inhibitor. In an embodiment, the compound of formula (I) is used for treating lung cancer in combination with a PI3K/mTOR inhibitor.

### DEFINITIONS

Unless otherwise specified, the below terms used herein are defined as follows:

The terms “treat”, “treatment” and “treating” include the reduction or amelioration of the progression, severity and/or duration of cancer, or the amelioration of one or more symptoms of cancer, resulting from the administration of the compound of formula (I). The terms “treat”, “treatment” and “treating” also include the reduction of the risk of recurrence of cancer or the delay or inhibition of the recurrence of cancer. In an embodiment, the terms “treat”, “treatment” and “treating” include the amelioration of at least one measurable physical parameter of cancer, such as growth of a tumor, not necessarily discernible by the patient. In other embodiments, the terms “treat”, “treatment” and “treating” includes the inhibition of the progression of cancer either physically by the stabilization of a discernible symptom, physiologically by the stabilization of a physical parameter, or both. In another embodiment, the terms “treat”, “treatment” and “treating” of cancer include the reduction or stabilization of tumor size or cancerous cell count, and/or delay of tumor formation.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features.

The term “EGFR” as used herein, refers to Epidermal Growth Factor Receptor (EGFR), a member of the type 1 subgroup of receptor tyrosine kinase family of growth factor receptors, which play critical roles in cellular growth, differentiation, and survival. Activation of these receptors typically occurs via specific ligand binding which results in hetero- or homodimerization between receptor family members, with subsequent autophosphorylation of the tyrosine kinase domain. Specific ligands which bind to EGFR include epidermal growth factor (EGF), transforming growth factor □(TGF□□, amphiregulin and some viral growth factors. Activation of EGFR triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation (Accession No. NP_{—}005219).

A number of human malignancies are associated with aberrant (mutated) or overexpression of EGFR and/or overexpression of its specific ligands (Gullick, Br. Med. Bull. (1991), 47:87-98; Modijtahedi and Dean, Int. J. Oncol. (1994), 4:277-96; Salomon, et al., Crit. Rev. Oncol. Hematol. (1995); 19:183-232, each of which is incorporated herein by reference). Aberrant or overexpression of EGFR has been associated with an adverse prognosis in a number of human cancers, including cancers of the head and neck, breast, colon, prostate, lung (e.g., NSCLC, adenocarcinoma and squamous lung cancer), ovaries, gastrointestinal tract (gastric, colon, pancreatic), kidneys, bladder, central nervous system (e.g., glioma), prostate, and gynecological carcinomas. In some instances, overexpression of tumor EGFR has been correlated with both chemoresistance and a poor prognosis (Lei, et al., Anticancer Res. (1999), 19:221-8; Veale, et al., Br. J. Cancer (1993); 68:162-5.

The term “EGFR inhibitor”, as used herein, includes any compound that disrupts EGFR production within a cell or disrupts activation of EGFR signaling in the cell activation of EGFR, leading to the Ras signaling cascade that results in uncontrolled cell proliferation. EGFR inhibitors include monoclonal antibodies that bind EGFR to inactivate it, and compounds that bind to the tyrosine kinase domain of EGFR to inhibit it. EGFR inhibitors include drugs such as erlotinib, gefitinib, and cetuximab. Particularly, erlotinib is described in U.S. Pat. Nos. 5,747,498, 6,900,221, 7,087,613, and RE41065. Trade names of certain EGFR inhibitors described herein include Tarceva®, Iressa®, and Erbitux®.

The KRAS oncogene (the cellular homolog of the Kirsten rat sarcoma virus gene, Accession No. NP_{—}203524) is a critical gene in the development of a variety of cancers, and the mutation status of this gene is an important characteristic of many cancers. Mutation status of the gene can provide diagnostic, prognostic and predictive information for several cancers. The KRAS gene is a member of a family of genes (KRAS, NRAS and HRAS). KRAS is a member of the RAS family of oncogenes, a collection of small guanosine triphosphate (GTP)-binding proteins that integrate extracellular cues and activate intracellular signaling pathways to regulate cell proliferation, differentiation, and survival. Gain-of-function mutations that confer transforming capacity are frequently observed in KRAS, predominantly arising as single amino acid substitutions at amino acid residues G12, G13 or Q61. Constitutive activation of KRAS leads to the persistent stimulation of downstream signaling pathways that promote tumorigenesis, including the RAF/MEK/ERK and PI3K/AKT/mTOR cascades. In NSCLC, KRAS mutations are highly prevalent (20-30%) and are associated with unfavorable clinical outcomes. Mutations in KRAS appear mutually exclusive with those in EGFR in NSCLC tumors; more importantly, they can account for primary resistance to targeted EGFR TKI therapies. Mutations in the KRAS gene are common in many types of cancer, including pancreatic cancer (˜65%), colon cancer (˜40%), lung cancer (˜20%) and ovarian cancer (˜15%).

A variety of laboratory methods have been utilized to detect mutations in the KRAS gene. See, e.g., Jimeno et al, KRAS mutations and sensitivity to epidermal growth factor receptor inhibitors in colorectal cancer: practical application of patient selection. J. Clin. Oncol. 27, 1130-1135 (2009); Van Krieken et al. KRAS mutation testing for predicting response to anti-EGFR therapy for colorectal carcinoma: proposal for a European quality assurance program. Virchows Archiv. 453, 417-431 (2008). Most methods include the use of PCR to amplify the appropriate region of the KRAS gene, including exons 2 and 3, and then utilize different methods to distinguish wild-type from mutant sequences in key codons, such as 12 and 13. The detection methods include nucleic acid sequencing, allele-specific PCR methods, single-strand conformational polymorphism analysis, melt-curve analysis, probe hybridization and others. The main features for consideration for these molecular techniques are the ability to distinguish the appropriate spectrum of variants at the codons of interest and the sensitivity or limit of detection (LOD) for mutant alleles. Both of these parameters are important, given the fact that tumors may be very heterogeneous, both with regard to the percentage of tumor cells within a given tissue and the potential for genetic heterogeneity.

More over, many methods have also been developed for KRAS mutation analysis to address various specific issues, related to increased analytical sensitivity, and they include allele-specific PCR using amplification refractory mutation system (ARMS) technology or coamplification at a lower denaturation temperature-PCR methods, pyrosequencing approaches and real-time PCR methods that use specific probe technologies, such as peptide nucleic acids. See, e.g., Pritchard et al, COLD-PCR enhanced melting curve analysis improves diagnostic accuracy for KRAS mutations in colorectal carcinoma. BMC Clin. Pathol. 10, 1-10 (2010); Weichart et al, KRAS genotyping of paraffin-embedded colorectal cancer tissue in routine diagnostics: comparison of methods and impact of histology. J. Mol. Diagn. 12, 35-42 (2010); Oliner et al, A comparability study of 5 commercial KRAS tests. Diagn. Pathol. 5, 23-29 (2010); Ogino et al, Brahmandan M et al. Sensitive sequencing method for KRAS mutation detection by pyrosequencing. J. Mol. Diagn. 4, 413-421 (2005).

There are several examples of laboratory-developed tests (LDTs) for detecting KRAS mutations, as well as a series of kits for research and for use in clinical diagnostics. For example, the TheraScreen® assay (DxS, Manchester, UK) is a CE-marked kit intended for the detection and qualitative assessment of seven somatic mutations in the KRAS gene, to aid clinicians in the identification of colorectal cancer patients who may benefit from anti-EGFR therapies, such as panitumumab and cetuximab. This assay uses an amplification refractory mutation system (ARMS), which is a version of allele-specific PCR; and detection of amplification products with Scorpion™ probes. See, e.g., TheraScreen® Package Insert, DsX, Manchester, UK (2009); Whitehall et al, A multicenter blinded study to evaluate KRAS mutation testing methodologies in the clinical setting. J. Mol. Diagn. 11, 543-552 (2009); Oliner et al, A comparability study of 5 commercial KRAS tests. Diagn. Pathol. 5, 23-29 (2010).

In addition, the European Society of Pathology (ESP), to help evaluate the reliability of KRAS mutation testing, has established a quality-assurance program for KRAS mutation analysis in colorectal cancers at http://kras.equascheme.org.

The ALK (anaplastic lymphoma kinase, Accession No. NP_{—}004295) RTK (receptor tyrosine kinase) was originally identified as a member of the insulin receptor subfamily of RTKs that acquires transforming capability when truncated and fused to NPM (nucleophosmin) in the t(2;5) chromosomal rearrangement associated with ALCL (anaplastic large cell lymphoma). To date, many chromosomal rearrangements leading to enhanced ALK activity have been described and are implicated in a number of cancer types. Recent reports of the EML4 (echinoderm microtubule-associated protein like 4)-ALK oncoprotein in NSCLC, together with the identification of activating point mutations in neuroblastoma, have highlighted ALK as a significant player and target for drug development in cancer. Representative ALK abnormalities (or “ALK+”) include EML4-ALK fusions, KIF5B-ALK fusions, TGF-ALK fusions, NPM-ALK fusions, and ALK point mutations.

The following two assays are presented for general information about detection and identification of ALK alterations, mutations or rearrangements in an ALK gene or gene product. These types of assays were also used in obtaining the results in Examples 1 and 2 herein.

The EML4/ALK assay detects eight known fusion variants and other undefined variants, in conjunction with measuring expression of wild type EML4 and ALK 5′ and 3′.

Lung cancer is the most common and deadly form of cancer in the USA, with a 5-year survival rate of approximately 15 percent. A subset of NSCLC patients have translocations which fuse the 5′ end of the EML4 gene to the 3′ end of the ALK gene creating an activated ALK oncogene. The incidence of ALK activation in NSCLC is low (2-7 percent), but it may be as high as 13 percent in patients with adenocarcinoma, no or a light history of smoking, younger age, and WT EGFR and KRAS genes. There are several other adenocarcinomas for which the ALK activation is relevant: breast, bladder, head & neck, and colon. Of particular interest, 5% of primary and metastatic melanoma patients harbor the translocation as well.

The EML4/ALK fusion protein displays constitutive ALK kinase activity, which can be targeted with ALK kinase inhibitors. The presence of an EML4/ALK translocation predicts a favorable response to ALK inhibitor therapy.

The quantitative Nuclease Protection Assay (gNPA™) is a multiplexed, lysis only assay of mRNA (53-58) that can also measure DNA and miRNA. What sets qNPA apart from other assays is that it does not require extraction of the DNA or RNA, but rather uses directly lysed samples. This permits high sample throughput, combined with the simultaneous measurement of DNA, mRNA and miRNA from the same lysate, and if necessary, on the same array.

qNPA also is very precise, with average whole assay CV's from tissues <10%, which means changes <1.2-fold can be detected, p<0.05. It is currently available as a low cost array plate-based assay measuring up to 47 genes/well.

Genetics: Multiple inversions on chromosome 2p generate in-frame fusions of the EML4 and ALK genes. While the breakpoints of EML4 can vary (fusion at exons 2, 6, 13, 14, 15, 18, and 20), the breakpoint of ALK occurs consistently at exon 20, 5′ of the kinase domain. The majority (˜70 percent) of translocations involve EML4 exon 13 (variant 1) or EML4 exon 6a/b (variant 3a/b). Due to close proximity of the EML4 and ALK genes, thus the small inversions, detection of some EML4/ALK variants is challenging with commercially available ALK break-apart FISH probes.

Product Format: The initial product is based upon the qNPA ArrayPlate format, either in 47 or 16 spot format as appropriate and dictated by the number of analytes to be tested with the ALK array.

Components: Kits are all inclusive with step-by-step instructions for ease of use.

Sample Type: Cell Lines, Blood, Purified RNA or FFPE

Intended Uses

The intended use for this product is to detect any of the specified expression wild types and fusion variants of ALK and EML4/ALK.

These are as follows:

WT: ALK—5′

WT: ALK—3′

Fusion: EML4/ALK-variant 1

Fusion: EML4/ALK-variant 2

Fusion: EML4/ALK-variant 3a

Fusion: EML4/ALK-variant 3b

Fusion: EML4/ALK-variant 4

Fusion: EML4/ALK-variant 5a

Fusion: EML4/ALK-variant 5b

Fusion: EML4/ALK-variant 6

Fusion: KIF5B-ALK

Fusion: TFG-ALK

WT: EML4-5′

WT: KIF5B-5′

WT: TFG-5″

Insight ALK Screen is an RT-qPCR assay that detects the presence of ALK fusions and upregulation of ALK wild type (which is abnormal in adult tissue outside the central nervous system and can be indicative of ALK-driven disease). The assay uses a three tube reaction series (plus controls) to measure expression of the extracellular segment of ALK (ALK WT), ALK kinase domain expression (ALK Kinase), and expression of an internal reference gene, Cytochrome c oxidase subunit 5B (COX5B). By focusing on relative expression of the ALK gene, Insight ALK Screen can more accurately detect the presence of ALK fusions than a variant-specific PCR approach that targets the 10+ unique 5′ gene partners, such as EML4.

Methods and procedures for the detection of wild type ALK and NPM-ALK fusions can be found in U.S. Pat. Nos. 5,529,925 and 5,770,421.

As used herein, a “subject with a mutation” in KRAS, ALK, EGFR, or other gene associated with cancer, or a “subject with a cancer with a mutation” in KRAS, ALK, EGFR, or other gene associated with cancer, and the like, are understood as a subject having cancer, wherein the tumor has at least one alteration (e.g., 1, 2, 3, 4, 5, 6, 7, 8, 9, 10, or more) in the indicated gene from the wild-type sequence in the gene and/or transcriptional, translational, and/or splicing control regions of the gene that result in the cell becoming cancerous, e.g., developing characteristics such as uncontrolled proliferation, immortality, metastatic potential, rapid growth and proliferation rate, decreased cell death/apoptosis, and certain characteristic morphological features. Mutations include, for example, insertions, deletions, truncations, point mutations, and translocations. Mutations within a gene product can result in constituent activation of the gene product. Mutations that include alterations in transcriptional, translational, or splicing control regions can result in aberrant expression, typically over-expression, of a wild-type gene product. It is understood that not all gene mutations, even in oncogenes, result in a cell becoming cancerous. Mutations that result in oncogenesis are well known in the art. Methods to test mutations for oncogenic activity are well known in the art.

A mutation can be detected using any of a number of known methods in the art. The specific method to detect the mutation will depend, for example, on the type of mutation to be detected. For example, alterations in nucleic acid sequences can be easily detected using polymerase chain reaction and fluorescence in situ hybridization methods (FISH). Protein expression levels can be detected, for example, using immunohistochemistry. An aberrant expression level of a wild-type protein can be used as a surrogate for detection of a mutation in a transcriptional, translational, and/or splicing control regions of the gene without direct detection of the specific genetic change in the nucleic acid in the subject sample. The specific method of detection of the mutation is not a limitation of the invention. Methods to compare protein expression levels to appropriate controls are well known in the art.

In a preferred embodiment, when multiple tests are used to detect a mutation and one is positive, the mutation is considered to be present. The methods do not require that multiple assays be performed to detect a mutation.

As used herein, and in the art, an “ALK+” tumor or cancer is understood as a tumor or cancer that has a mutation such that ALK is overexpressed and causes a cancerous phenotype in the cell.

As used herein, a subject with a “wild-type” KRAS, ALK, EGFR, or other gene associated with cancer, or a “subject with a cancer with a wild-type” KRAS, ALK, EGFR, or other gene associated with cancer, and the like, are understood as a subject suffering from cancer, wherein the tumor does not have any significant alterations (i.e., alterations that result in a change of function) in the indicated gene from the native sequence in the gene and/or transcriptional, translational, and/or splicing control regions of the native gene that result in the cell becoming cancerous, e.g., developing characteristics such as uncontrolled proliferation, immortality, metastatic potential, rapid growth and proliferation rate, decreased cell death/apoptosis, and certain characteristic morphological features. As used herein, a “wild-type” gene is expressed at a level that does not result in the cell becoming cancerous.

As used herein, and in the art, a “HER2 positive” tumor or cancer is a tumor or cancer that expresses a wild-type level of HER2. Loss of HER2 expression is associated with a cancer phenotype.

As used herein, and in the art, an “estrogen receptor positive” or “ER positive” tumor or cancer is a tumor or cancer that expresses a wild-type level of estrogen receptor (ER). Loss of ER expression is associated with a cancer phenotype.

As used herein, and in the art, a “progesterone receptor positive” or “PR positive” tumor or cancer is a tumor or cancer that expresses a wild-type level of progesterone receptor (PR). Loss of PR expression is associated with a cancer phenotype.

Mutations or protein expression levels are preferably detected in a subject sample from the cancer tissue or tumor tissue, e.g., cells, extracellular matrix, and other naturally occurring components associated with the tumor. The mutation or expression level can be detected in a biopsy sample or in a surgical sample after resection of the tumor. The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In an embodiment, the sample is removed from the subject. In a particular embodiment, the sample is urine or serum. In an embodiment, the sample comprises cells. In another embodiment, the sample does not comprise cells. In certain embodiments, the sample can be the portion of the subject that is imaged. Samples are typically removed from the subject prior to analysis; however, tumor samples can be analyzed in the subject, for example, using imaging or other detection methods.

As used herein, the terms “identify” or “select” refer to a choice in preference to another. In other words, to identify a subject or select a subject is to perform the active step of picking out that particular subject from a group and confirming the identity of the subject by name or other distinguishing feature. With respect to the instant invention, it is understood that identifying a subject or selecting a subject as having one or more mutations in one or more genes of interest, having a wild-type gene, or having a change in the expression level of a protein, and can include any of a number of acts including, but not limited to, performing a test and observing a result that is indicative of a subject having a specific mutation; reviewing a test result of a subject and identifying the subject as having a specific mutation; reviewing documentation on a subject stating that the subject has a specific mutation and identifying the subject as the one discussed in the documentation by confirming the identity of the subject e.g., by an identification card, hospital bracelet, asking the subject for his/her name and/or other personal information to confirm the subjects identity.

As used herein, the term “refractory” cancer or tumor is understood as a malignancy which is either initially unresponsive to chemo- or radiation therapy, or which becomes unresponsive over time. A cancer refractory to on intervention may not be refractory to all interventions. A refractory cancer is typically not amenable to treatment with surgical interventions.

As used herein, “relapse” is understood as the return of a cancer or the signs and symptoms of a cancer after a period of improvement.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

### Methods of Detection of Mutations

As already indicated, the methods and procedures for the detections and/or identifications of EGFR, KRAS, and/or ALK over-expressions and/or mutations are known in the literature and can be easily carried out by a skilled person. See, e.g., U.S. Pat. No. 7,700,339; U.S. Patent Application Publication No. US2011/0110923; Palmer et al, Biochem. J. (2009), 345-361; Koivunen et al, Clin. Can. Res., 2008, 14, 4275-4283; Anderson, Expert Rev. Mol. Diagn. 11(6), 635-642 (2011); Pinto et al, Cancer Genetics 204 (2011), 439-446; Rekhtman et al; Clin Cancer Res 2012; 18:1167-1176; Massarelli et al, Clin Cancer Res 2007; 13:2890-2896; Lamy et al, Modern Pathology (2011) 24, 1090-1100; Balschun et al, Expert Rev. Mol. Diagn. 11(8), 799-802 (2011); Vakiani et al, J Pathol 2011; 223, 219-229; Okudela et al, Pathology International 2010; 60: 651-660; John et al, Oncogene (2009) 28, S14-S23; and the references cited in the-above identified references. Thresholds of increased expression that constitute an EGFR mutation or an ALK mutation are well known in the art. Moreover, it is generally recognized that once an EGFR mutation is detected in a cancer, the KRAS mutation will be eliminated in the same cancer. Put reversely, if a KRAS mutation is positively identified in a cancer from a subject, it is then not necessary to engage in any further EGFR related identification. Similar principle can be applied to an ALK mutation in a cancer. That is if there is an ALK mutation detected in a cancer, it is extremely rare that an EGFR or KRAS mutation will be implicated. Stated another way, once an ALK mutation is positively identified in a cancer, no further identification is necessary either for EGFR mutation or for KRAS mutation in the same cancer.

Methods of detection of expression levels of ER, PR, and HER2 are well known in the art. Thresholds of expression to that constitute ER, PR, and HER2 status are also well known in the art.

As used herein, “detecting”, “detection” and the like are understood that an assay performed for identification of a specific analyte in a sample, e.g., a gene or gene product with a mutation, or the expression level of a gene or gene product in a sample, typically as compared to an appropriate control cell or tissue. The specific method of detection used is not a limitation of the invention. The detection method will typically include comparison to an appropriate control sample.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level of signal detected or protein expression in a control sample that consists of a group of measurements may be determined, e.g., based on any appropriate statistical measure, such as, for example, measures of central tendency including average, median, or modal values.

### Chemotherapeutic Agents

In certain embodiments, the compound of formula (I) is administered with one or more additional chemotherapeutic agents.

The taxanes are anti-cancer agents that include paclitaxel (Taxol®) and docetaxel (Taxotere®). Both drugs have proved to be effective in the treatment of a variety of solid tumors including breast, ovarian, lung, and bladder cancers. Thus, the term “paclitaxel analog” is defined herein to mean a compound which has the basic paclitaxel skeleton and which stabilizes microtubule formation. Many analogs of paclitaxel are known, including docetaxel. In addition, a paclitaxel analog can also be bonded to or be pendent from a pharmaceutically acceptable polymer, such as a polyacrylamide. The term “paclitaxel analog”, as it is used herein, includes such polymer linked taxanes.

The term “vascular endothelial growth factor inhibitor” or “VEGF inhibitor” includes any compounds that disrupt the function of vascular endothelial growth factor A (VEGF) production within a cell. VEGF inhibitors are another class of anticancer agents. VEGF inhibitors include drugs such as bevacizumab (Avastin®), sunitinib (Sutent®), and sorafenib (Nexavar®). Examples of VEGF receptor inhibitors include sunitinib and sorafenib. Monoclonal antibody therapies, such as bevacizumab, that block VEGF are described in U.S. Pat. Nos. 6,884,879, 7,060,269, and 7,297,334.

The dosages of other anti-cancer agents, which have been or are currently being used to prevent, treat, manage, or ameliorate disorders, such cancer, or one or more symptoms thereof can be used in the combination therapies of the invention. Preferably, dosages lower than those which have been or are currently being used to prevent, treat, manage, or ameliorate cancer, or one or more symptoms thereof, are used in the combination therapies of the invention. The recommended dosages of agents currently used for the prevention, treatment, management, or amelioration of cancer, or one or more symptoms thereof, can obtained from any reference in the art including, but not limited to, Hardman et al., eds., 1996, Goodman & Gilman's The Pharmacological Basis Of Basis Of Therapeutics 9^{th} Ed, Mc-Graw-Hill, N.Y.; Physician's Desk Reference (PDR) 57^{th} Ed., 2003, Medical Economics Co., Inc., Montvale, N.J.

An “effective amount” is that amount sufficient to treat a disease in a subject. A therapeutically effective amount can be administered in one or more administrations.

The term “effective amount” includes an amount of the compound of formula (I) which is sufficient to treat the cancer, to reduce or ameliorate the severity, duration, or progression of cancer, to retard or halt the advancement of cancer, to cause the regression of cancer, to delay the recurrence, development, onset, or progression of a symptom associated with cancer, or to enhance or improve the therapeutic effect(s) of another therapy. For example, an effective amount can induce, for example, a complete response, a partial response, or stable disease; as determined, for example, using RESIST criteria.

An “effective amount” of a therapeutic agent produces a desired response. Having a positive response to treatment with a therapeutic agent is understood as having a decrease in at least one sign or symptom of a disease or condition (e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). It is understood that an “effective amount” need not be curative.

An effective amount of a compound of formula (I) is understood as an amount of the compound of formula (I) to improves outcome relative to an appropriate control group, e.g., an untreated group, a group treated with a combination of therapies not including the compound of formula (I). Methods to select appropriate control groups and to perform comparative analyses are within the ability of those of skill in the art.

The precise amount of compound administered to provide an “effective amount” of the compound of formula (I) to the subject will depend on the mode of administration, the type and severity of the cancer and on the characteristics of the subject, such as general health, age, sex, body weight and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th ed., 2003).

The dosage of an individual agent used in combination therapy may be equal to or lower than the dose of an individual therapeutic agent when given independently to treat, manage, or ameliorate a disease or disorder, or one or more symptoms thereof. In one embodiment, the disease or disorder being treated with a combination therapy is a triple-negative breast cancer.

In an embodiment, the amount of the compound of formula (I) administered is from about 2 mg/m² to about 500 mg/m², for example, from about 100 mg/m² to about 500 mg/m², from about 125 mg/m² to about 500 mg/m², from about 150 mg/m² to about 500 mg/m² or from about 175 mg/m² to about 500 mg/m². In an embodiment, the amount of the compound of formula (I) administered is about 100 mg/m² to about 300 mg/m², from about 125 mg/m² to about 300 mg/m², from about 150 mg/m² to about 300 mg/m² or from about 175 mg/m² to about 300 mg/m². In some embodiments, the amount of the compound of formula (I) administered is about 2 mg/m², 4 mg/m², about 7 mg/m², about 10 mg/m², about 14 mg/m², about 19 mg/m², about 23 mg/m², about 25 mg/m², about 33 mg/m², about 35 mg/m², about 40 mg/m², about 48 mg/m², about 49 mg/m², about 50 mg/m², about 65 mg/m², about 75 mg/m², about 86 mg/m², about 100 mg/m², about 110 mg/m², about 114 mg/m², about 120 mg/m², about 144 mg/m², about 150 mg/m², about 173 mg/m², about 180 mg/m², about 200 mg/m², about 216 mg/m² or about 259 mg/m².

The language “twice-weekly” includes administration of a compound of formula (I) two times in about 7 days. For example, the first dose of the compound of formula (I) is administered on day 1, and the second dose of the compound of formula (I) may be administered on day 2, day 3, day 4, day 5, day 6 or day 7. In some embodiments, the twice-weekly administration occurs on days 1 and 3 or days 1 and 4.

In some embodiments, the compound of formula (I) is cyclically administered twice-weekly. For example, the compound of formula (I) is administered for a first period of time, followed by a “dose-free” period, then administered for a second period of time. The language “dose-free” includes the period of time in between the first dosing period and the second dosing period in which no compound of formula (I) is administered to the subject. A preferred cycle is administering the compound of formula (I) at a dose described above two times during the week for three consecutive weeks followed by one dose-free week. This cycle is then repeated, as described below.

The language “one cycle” includes the first period of time during which the compound of formula (I) is administered, followed by a dose-free period of time. The dosing cycle can be repeated and one of skill in the art will be able to determine the appropriate length of time for such a cyclical dosing regimen. In an embodiment, the cycle is repeated at least once. In an embodiment, the cycle is repeated two or more times. In an embodiment, the cycle is repeated 3, 4, 5, 6, 7, 8, 9, 10, 11, 12, 13, 14, 15 or more times, or as many times as medically necessary as determined by one of skill in the art, e.g., as long as the subject exhibits a response with no dose limiting toxicities. In an embodiment, the cycle is repeated until the patient has been determined to be in partial remission (e.g., 50% or greater reduction in the measurable parameters of tumor growth) or complete remission (e.g., absence of cancer). One of skill in the art can determine a patient's remission status using routine methods well known in the art.

The language “pharmaceutically acceptable salt” includes salts prepared from a compound of formula (I) by reacting the phenolic functional groups and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. A pharmaceutically acceptable salt can also be formed by reacting the amine functional groups and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, palmoic acid and p-toluenesulfonic acid.

The language “tautomer of a compound of formula (I)” includes all tautomeric forms of the compound of formula (I). In an embodiment, the tautomer of the compound of formula (I) is the compound of formula (Ia):

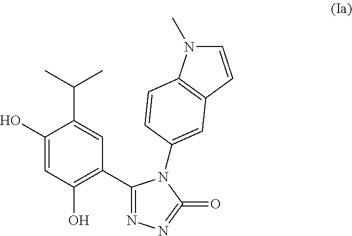

or a pharmaceutically acceptable salt thereof.

As used herein, the term “in combination” refers to the use of more than one therapeutic agent (e.g., 2, 3, 4, 5, 6, 7, 8, 9, 10, or more). The use of the term “in combination” does not restrict the order in which the therapeutic agents are administered to a subject afflicted with cancer. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent or treatment, such as an anti-cancer agent, to a subject with cancer. In certain embodiments, one agent may be administered more frequently than the other agent such that multiple doses of one agent are administered for each dose of the other agent(s).

In an embodiment, the method comprises administering to the subject with a cancer with a KRAS mutation an effective amount of a combination of a compound of formula (I), or a tautomer or pharmaceutically acceptable salt thereof, and one or more of BEZ235, AZD6244, AZD8055, SN-38, gemcitabine, camptothecin, docetaxel, cisplatin, oxaliplatin, crizotinib, paclitaxel, trastuzumab, and pemetrexed. In an embodiment, the method is in combination with BEZ235. In an embodiment, the combination is with AZD6244. In an embodiment, the combination is with AZD8055. In an embodiment, the combination is with SN-38. In an embodiment, the combination is with gemcitabine. In an embodiment, the combination is with camptothecin. In an embodiment, the combination is with docetaxel. In an embodiment, the combination is with cisplatin. In an embodiment, the combination is with oxaliplatin. In an embodiment, the combination is with crizotinib. In an embodiment, the combination is with trastuzumab. In an embodiment, the combination is with pemetrexed.

In an embodiment, the compound of formula (I) may be used in combination with one or more additional anti-cancer agents for treatment of a subject with a NSCLC with a KRAS mutation. In an embodiment, the method comprises administering to the subject with NSCLC with a KRAS mutation an effective amount of a combination of a compound of formula (I), or a tautomer or pharmaceutically acceptable salt thereof, and one or more of BEZ235, AZD6244, AZD8055, SN-38, gemcitabine, camptothecin, docetaxel, cisplatin, oxaliplatin, crizotinib, paclitaxel, trastuzumab, or pemetrexed. In an embodiment, the combination is with BEZ235. In an embodiment, the combination is with AZD6244. In an embodiment, the combination is with AZD8055. In an embodiment, the combination is with SN-38. In an embodiment, the combination is with gemcitabine. In an embodiment, the combination is with camptothecin. In an embodiment, the combination is with docetaxel. In an embodiment, the combination is with cisplatin. In an embodiment, the combination is with oxaliplatin. In an embodiment, the combination is with crizotinib. In an embodiment, the combination is with trastuzumab. In an embodiment, the combination is with pemetrexed.

In an embodiment, the method comprises administering to the subject with an ALK+ cancer an effective amount of a combination of a compound of formula (I), or a tautomer or pharmaceutically acceptable salt thereof, and one or more of BEZ235, AZD6244, AZD8055, SN-38, gemcitabine, camptothecin, docetaxel, cisplatin, oxaliplatin, crizotinib, paclitaxel, trastuzumab, or pemetrexed. In an embodiment, the combination is with BEZ235. In an embodiment, the combination is with AZD6244. In an embodiment, the combination is with AZD8055. In an embodiment, the combination is with SN-38. In an embodiment, the combination is with gemcitabine. In an embodiment, the combination is with camptothecin. In an embodiment, the combination is with docetaxel. In an embodiment, the combination is with cisplatin. In an embodiment, the combination is with oxaliplatin. In an embodiment, the combination is with crizotinib. In an embodiment, the combination is with trastuzumab. In an embodiment, the combination is with pemetrexed.

In an embodiment, the compound of formula (I) may be administered for treating ALK+ NSCLC in a subject in combination with one or more additional anti-cancer agents. In an embodiment, the method comprises administering to the subject with ALK+ NSCLC an effective amount of a combination of a compound of formula (I), or a tautomer or pharmaceutically acceptable salt thereof, and one or more of BEZ235, AZD6244, AZD8055, SN-38, gemcitabine, camptothecin, docetaxel, cisplatin, oxaliplatin, crizotinib, paclitaxel, trastuzumab, or pemetrexed. In an embodiment, the combination with BEZ235. In an embodiment, the combination is with AZD6244. In an embodiment, the combination is with AZD8055. In an embodiment, the combination is with SN-38. In an embodiment, the combination is with gemcitabine. In an embodiment, the combination is with camptothecin. In an embodiment, the combination is with docetaxel. In an embodiment, the combination is with cisplatin. In an embodiment, the combination is with oxaliplatin. In an embodiment, the combination is with crizotinib. In an embodiment, the combination with trastuzumab. In an embodiment, the combination is with pemetrexed.

In an embodiment, the one or more additional anti-cancer agents include one or more of VEGF inhibitors (e.g., bevacizumab, sunitinib, or sorafenib), EGFR inhibitors (e.g., erlotinib, gefitinib or cetuximab), tyrosine kinase inhibitors (e.g., imatinib), proteosome inhibitors (e.g., bortezomib), taxanes (e.g., paclitaxel and paclitaxel analogues), and ALK inhibitors (e.g., crizotinib). In an embodiment, the additional anticancer drug is trastuzumab.

In an embodiment, the compound of formula (I) is used for treating lung cancer in combination with a MEK inhibitor such as such as AZD6244 (also called ARR^{y}-142886), PD098059, PD184352, PD0325901, PD 318088, or U0126. In an embodiment, the compound of formula (I) is used for treating lung cancer in combination with a PI3K/mTOR inhibitor such as [5-[2,4-bis((3S)-3-methylmorpholin-4-yl)pyrido[5,6-e]pyrimidin-7-yl]-2-methoxyphenyl]methanol (AZD8055), 2-methyl-2-[4-[3-methyl-2-oxo-8-(quinolin-3-yl)-2,3-dihydroimidazo[4,5-c]quinolin-1-yl]phenyl]propionitrile (BEZ235, or NVP-BEZ235), deforolimus (MK-8669), everolimus (RAD001), (5Z)-5-[[4-(4-pyridinyl)-6-quinolinyl]methylene]-2,4-thiazolidinedione (GSK1059615), 5-[2-[(2R,6S)-2,6-dimethyl-4-morpholinyl]-4-(4-morpholinyl)pyrido[2,3-d]pyrimidin-7-yl]-2-methoxy-benzenemethanol (KU-0063794), 6H-8-(1-hydroxyethyl)-2-methoxy-3-[(4-methoxyphenyl)methoxy]-Dibenzo[b,d]pyran-6-one, (Palomid 529, or P529), 3-[4-(4-morpholinyl)pyrido[3′,2′:4,5]furo[3,2-d]pyrimidin-2-yl]-phenol (PI-103), 2-[4-amino-1-(1-methylethyl)-1H-pyrazolo[3,4-d]pyrimidin-3-yl]-1H-Indol-5-o1 (PP242), rapamycin (Sirolimus), 4-[6-[4-[(methoxycarbonyl)amino]phenyl]-4-(4-morpholiny)-1H-pyrazolo[3,4-d]pyrimidin-1-y1]-1-piperidinecarboxylic acid, methyl ester (WYE-354), or XL765 (SAR245409). In an embodiment, the compound of formula (I) is used for treating lung cancer in combination with a MEK inhibitor and a PI3K/mTOR inhibitor.

The compound of formula (I) and optionally, one or more additional anti-cancer agents, can be administered to a subject by routes known to one of skill in the art. Examples of routes of administration include, but are not limited to, parenteral, e.g., intravenous, intradermal, subcutaneous, oral, intranasal (e.g., inhalation), transdermal, topical, transmucosal, and rectal administration. The agents can be administered by different routes of administration.

The compound of formula (I), and optionally, one or more additional anti-cancer agents, may be formulated with a pharmaceutically acceptable carrier, diluent, or excipient as a pharmaceutical composition. Pharmaceutical compositions and dosage forms of the invention comprise one or more active ingredients in relative amounts and formulated in such a way that a given pharmaceutical composition or dosage form can be used to treat cancer. Administration in combination does not require co-formulation.

A pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In some embodiments, the compound of formula (I) is formulated at a concentration of 8 mg/mL in 90% v/v PEG 300 and 10% v/v Polysorbate 80 for intravenous administration.

The invention also provides a compound of structural formula (I) or a pharmaceutically acceptable salt thereof for use in treating a subject with a NSCLC with a KRAS mutation. The invention also provides a compound of structural formula (I) or a pharmaceutically acceptable salt thereof for use in treating a subject with NSCLC with a KRAS mutation in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of a compound of structural formula (I) or a pharmaceutically acceptable salt thereof for the manufacture of a medicament for the treatment of a subject with a cancer with an EGFR mutation. The invention further provides the use of a compound of structural formula (I) or a pharmaceutically acceptable salt thereof for the manufacture of a medicament for the treatment of a subject with a cancer with an EGFR mutaiton in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of a compound of structural formula (I) or a pharmaceutically acceptable salt thereof for the manufacture of a medicament for the treatment of a subject with a NSCLC with an EGFR mutation. The invention further provides the use of a compound of structural formula (I) or a pharmaceutically acceptable salt thereof for the manufacture of a medicament for the treatment of a subject with a NSCLC with an EGFR mutation in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides a compound of structural formula (I) or a pharmaceutically acceptable salt thereof for use in treating a subject with a cancer with an EGFR mutation. The invention also provides a compound of structural formula (I) or a pharmaceutically acceptable salt thereof for use in treating a subject with a cancer with an EGFR mutation in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides a compound of structural formula (I) or a pharmaceutically acceptable salt thereof for use in treating a subject with a NSCLC with an EGFR mutation. The invention also provides a compound of structural formula (I) or a pharmaceutically acceptable salt thereof for use in treating a subject with a NSCLC with an EGFR mutation in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of a compound of structural formula (I) or a pharmaceutically acceptable salt thereof for the manufacture of a medicament for the treatment of a subject with an ALK+ cancer. The invention further provides the use of a compound of structural formula (I) or a pharmaceutically acceptable salt thereof for the manufacture of a medicament for the treatment of a subject with an ALK+ cancer in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of a compound of structural formula (I) or a pharmaceutically acceptable salt thereof for the manufacture of a medicament for the treatment of a subject with an ALK+ NSCLC. The invention further provides the use of a compound of structural formula (I) or a pharmaceutically acceptable salt thereof for the manufacture of a medicament for the treatment of a subject with an ALK+ NSCLC in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides a compound of structural formula (I) or a pharmaceutically acceptable salt thereof for use in treating a subject with an ALK+ cancer. The invention also provides a compound of structural formula (I) or a pharmaceutically acceptable salt thereof for use in treating a subject with an ALK+ cancer in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides a compound of structural formula (I) or a pharmaceutically acceptable salt thereof for use in treating a subject with an ALK+NSCLC. The invention also provides a compound of structural formula (I) or a pharmaceutically acceptable salt thereof for use in treating a subject with an ALK+NSCLC in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of a compound of structural formula (I) or a pharmaceutically acceptable salt thereof for the manufacture of a medicament for the treatment of a subject with triple negative breast cancer. The invention further provides the use of a compound of structural formula (I) or a pharmaceutically acceptable salt thereof for the manufacture of a medicament for the treatment of a subject with triple negative breast cancer in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides a compound of structural formula (I) or a pharmaceutically acceptable salt thereof for use in treating a subject with triple negative breast cancer. The invention also provides a compound of structural formula (I) or a pharmaceutically acceptable salt thereof for use in treating a subject with triple negative breast cancer in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention is illustrated by the following examples, which are not intended to be limiting in any way.

### EXAMPLES

### Example 1

### A Phase I Dose Escalation Study of the Compound of Formula (I) (Ganetespib) in Twice-Weekly Administration in Patients with Solid Tumors

An open-label Phase 1 dose-escalation study in subjects with solid tumors was performed. The first cohort consisted of three subjects who received 2 mg/m² of compound of formula (I) during a 1-hour infusion 2 times per week (e.g., [Monday, Thursday] or [Tuesday, Friday]) for three consecutive weeks followed by a 1 week dose-free interval. The first infusion for the first three subjects was staggered by a minimum of 5 days between subjects. This staggered enrollment scheme was followed for the first cohort only. Subjects tolerating compound of formula (I) continued treatment past week 8 until disease progression as long as the re-treatment criteria continued to be met.

Subsequent cohorts were originally planned to receive escalating doses of 4, 7, 10, 14, 19, 25, 33, 40 and 48 mg/m², provided that the previous dose was well-tolerated during cycle 1 (week 1-4). The dose escalation scheme was updated to 25, 50, 75 and 100 mg/m², provided that the previous dose was well-tolerated during cycle 1. Following the completion of enrollment at 50 mg/m² twice per week, subsequent cohorts were to be treated at 100 mg/m² (100% increase above prior dose) and 120 mg/m² (20% increase above prior dose), with further dose increments to be approximately 20% over the previous dose level, until the maximum tolerated dose (MTD) was determined. Enrollment was completed at 100 mg/m² and the next doses planned were 120 mg/m² and 144 mg/m².

There had to be at least three evaluable subjects in a cohort before dose escalation could occur. An evaluable subject was defined as one who had received at least 5 of 6 doses of compound of formula (I) during cycle 1 and had a subsequent follow up visit or experienced a dose limiting toxicity (DLT) after any dose. Once a subject experienced a DLT the cohort was expanded to six subjects. If only 1 of 6 subjects experienced a DLT, further dose escalation was allowed. However, if 2 of 3 or 2 of 6 subjects experienced a DLT, dose escalation terminated.

A subject's duration of participation included a 2-week screening period and two 4-week treatment cycles totaling approximately 10 weeks. However, at the investigator's discretion, subjects tolerating the compound of formula (I) continued treatment past week 8 until disease progression.

The subjects in this study had histologically- or cytologically-confirmed non-hematological malignancy that was metastatic or unresectable. The subjects were documented to be refractory to, or were not candidates for, current standard therapy.

The compound of formula (I) was formulated using 90% v/v PEG 300 and 10% v/v Polysorbate 80 at a concentration of 8 mg/mL and was packaged in a Type I glass amber vial, stoppered with a Fluorotec®-coated stopper, and sealed. Each vial had a deliverable volume of 12.5 mL (equivalent to 100 mg/vial). The formulation was further diluted with 5% dextrose for injection in infusion container (DEHP-free 500 mL) to a concentration range of 0.02 to 1.2 mg/mL and administered via infusion tubing (DEHP-free) with a 0.22 micron end filter over an hour to the patient. The dosing solution once prepared was administered within 3 hours. Eligible subjects received the drug during a 1-hour infusion 2 times per week for 3 consecutive weeks followed by a 1-week dose-free interval. The amount of the compound of formula (I) administered depended upon the cohort to which the subject was assigned and the subject's body surface area (BSA). This cycle was repeated for subjects tolerating the compound of formula (I) who did not experience disease progression.

Forty-one of 54 enrolled patients were assessable for response. A total of 13 patients discontinued prior to the Week 8 response assessment. Confirmed partial responses included 1 patient with melanoma and 1 patient with triple negative breast cancer. Fifteen (15) patients achieved stable disease.

### Example 2

### Efficacy of the Compound of Formula (I) in the Treatment of Triple Negative Breast Cancer Subject from Example 1

Triple-negative breast cancer (TNBC) represents 10-20% of all diagnosed breast cancer cases and tests negative for the presence of estrogen receptor (ER), progesterone receptor (PR), and the human epidermal growth factor receptor 2 (HER2). Therefore, this breast cancer subtype does not respond to hormonal therapy used to treat breast cancer, such as tamoxifen or aromatase inhibitors, or therapies that target HER2 receptors, such as Herceptin®. Triple-negative breast cancer is characterized as more aggressive than other breast cancer subtypes, disproportionately affects younger women, and is associated with a poorer 5-year survival rate of 77%, as compared to the 93% survival rate for other cancers. Triple-negative breast cancer is typically treated with a combination of therapies such as surgery, radiation therapy, and chemotherapy, however, early relapse and metastasis is common.

A 39-year old white female with triple negative breast cancer (with Stage III invasive ductal carcinoma) from Example 1 enrolled in a Phase I dose escalation study of compound of formula (I). The patient's disease had progressed on 7 prior chemotherapeutic regimens. The patient was administered 144 mg/m² of compound of formula (I) twice-weekly for 3 weeks, followed by 1 week dose-free. After 2 cycles, she demonstrated stable disease per Response Evaluation Criteria in Solid Tumors (RECIST). After 4 cycles, there was a documented 31% reduction in target lesion size (partial response). The treatment was interrupted due to brain metastases treated with whole brain radiation, but treatment with compound of formula (I) resumed in cycle 5. The patient tolerated the treatment well with mild/moderate toxicities.

### Example 3

### Efficacy of the Compound of Formula (I) in the Treatment of Non-Small Cell Lung Cancer (NSCLC) by KRAS, EGFR, and ALK Mutation Status

A Phase 2 clinical trial was performed to determine the efficacy of the compound of formula (I) in the treatment of NSCLC.

Patients with advanced NSCLC who failed prior treatments received 200 mg/m² of compound of formula (I) as a 1-hr infusion once weekly for 3 of a 4-wk cycle in a Simon two-stage study design assessing primary endpoint of PFS rate at 16 wks. Initial cohorts were defined by mutation status: A) EGFR mutation, KRAS wild-type B) EGFR wild-type, KRAS mutation C) EGFR wild-type, KRAS wild-type (WT). If ≧2/14 patients in A, B or C were progression-free at 16 weeks, enrollment increased to 23 patients for that cohort. Tumor response was assessed every 8 weeks. Cohort D was added to include 35 additional EGFR wild-type and KRAS wild-type patients with adenocarcinoma histology. FISH analysis for ALK translocation, was performed for Cohorts C and D.

There were 73 patients (31 M, 42 F; median age 62 yrs, range 28-82; ECOG 0-1; prior therapies range 1-10) that received a median of 2 cycles (range 1-12) of compound of formula (I) in cohorts A (14), B (17), and C+D (42). Adverse events reported in >20% of patients included diarrhea, fatigue, nausea, anorexia, constipation, and dyspnea and were generally grade 1-2.

In Cohort A, subjects with an EGFR mutation and a wild-type KRAS

In Cohort B, subjects with a wild-type EGFR and a KRAS mutation, greater than 60% of patients with NSCLC exhibited tumor shrinkage at 8 weeks, indicating that the compound of formula (I) is useful in the treatment of NSCLC with a KRAS mutation.

Expansion criteria were achieved for cohort C, including a durable partial response (PR) and seven patients with prolonged stable disease (≧16 weeks).

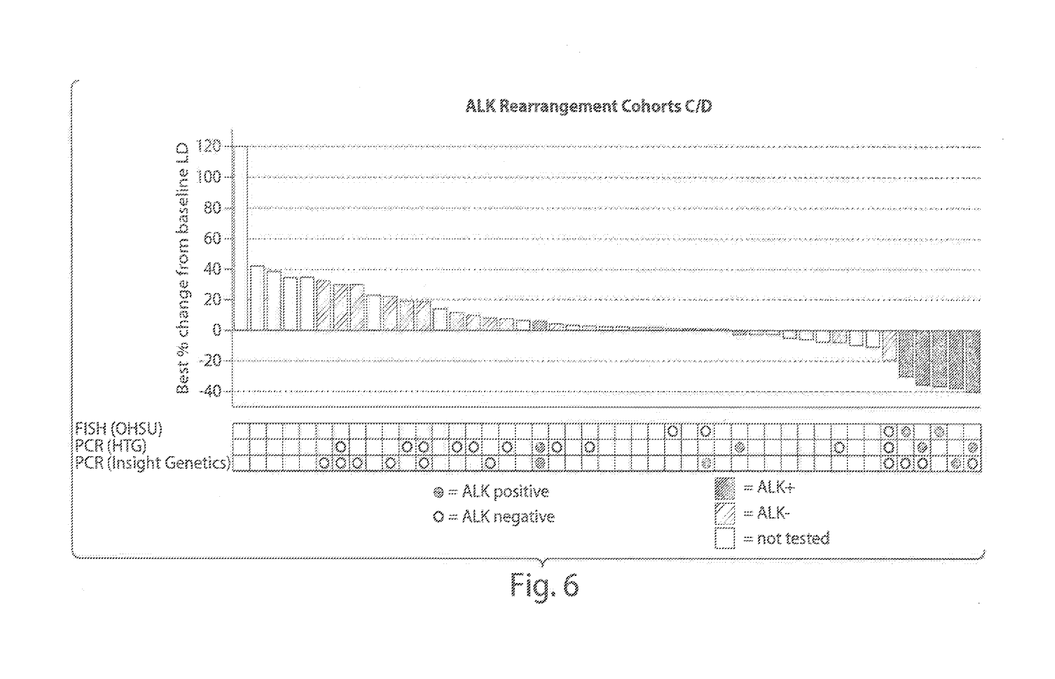

Of the 23 patients of cohorts C and D (EGFR wild-type, KRAS wild-type) in the Phase 2 trial tested for ALK translocation or rearrangement (ALK+), eight patients were ALK+ in at least one assay. Six of these eight ALK+ patients (75%) showed tumor shrinkage in target lesions. One ALK+ patient showed no change in tumor size, and one ALK+ patient achieved stable disease (tumor growth <20%). The disease control rate in this population was 7/8 (88%), and the objective response rate (complete response (CR)+ partial response (PR)) was 4/8 (50%) (see FIG. 6).

In summary, compound of formula (I) administered as a single-agent was well-tolerated in patients with NSCLC at 200 mg/m² once weekly without severe liver, ocular, cardiovascular or renal toxicity. Clinical activity was observed in patients with advanced NSCLC tumors with both a wild-type EGFR and a KRAS mutation; a wild-type EGFR and a wild-type KRAS. Clinical activity was observed in patients with ALK+ NSCLC tumors (i.e., tumors with an ALK mutation). This demonstrates the utility of the compound of formula (I) for the treatment of NSCLC with various mutations.

### Example 4

### Efficacy of the Compound of Formula (I) in a Phase 2 Study for the Treatment of Gastrointestinal Stromal Tumors (GIST)

A gastrointestinal stromal tumor (GIST) is a type of cancer that occurs in the gastrointestinal (GI or digestive) tract, including the esophagus, stomach, gall bladder, liver, small intestine, colon, and rectum. The American Cancer Society estimates 4,500 to 6,000 GIST cases are diagnosed each year in the United States. Although these tumors can start anywhere in the GI tract, they occur most often in the stomach (50% to 70%) or the small intestine (20% to 30%). Gastric cancer is second to lung cancer as the most lethal cancer worldwide, with 5-year survival rates in the range of 10% to 15%.

Patients with advanced (e.g., metastatic or unresectable) GIST following failure of prior therapy, e.g., imatinib or sunitinib, received compound of formula (I) (200 mg/m²) as a 1 hour IV infusion once per week for 3 weeks of a 28 day cycle. GIST status was assessed at 8 weeks per RECIST, until progression. In this Simon's 2 stage study design, if ≧4/23 patients in Stage 1 had clinical benefit (CR+PR+ stable disease (SD)≧16 wks) enrolment would continue with Stage 2. Hsp90 client protein levels were analyzed in biopsies pre-therapy and 24-48 h post-treatment with the compound of formula (I) in a subset of patients.

There were 26 patients (15 M, 11 F; median age 53 yrs, range 33-67; ECOG status 0-1; median 5 prior therapy regimens, range 3-12, wild-type platelet derived growth factor receptor alpha (PDGFRA)) that received a median of 2 cycles of compound of formula (I) (range 1-8). Adverse events reported in >20% of patients were generally NCl CTC grade 1-2 and included diarrhea, fatigue, nausea, vomiting, increased alkaline phosphatase, headache, insomnia, and abdominal pain. At the time of the analysis, 23 patients out of 26 patients in the intent to treat (ITT) population were evaluable. 12/23 evaluable patients had SD (4 SD≧16 wks, 8 SD≧8 wks), meeting formal criteria to enroll Stage 2. However, analysis of client proteins in paired tumor biopsies from 4 patients did not show prolonged inhibition of activated KIT or its downstream pathways.

In summary, compound of formula (I) given by once-weekly dosing was well-tolerated in patients with heavily pre-treated advanced GIST, with no evidence of severe liver, ocular, cardiac or renal toxicity. Disease stabilization was seen in a subset of patients. These results demonstrate the utility of the compound of formula (I) in the treatment of GIST.

### Example 5

### Efficacy of the Compound of Formula (I) in a Phase 2 Study for the treatment of solid tumors

A phase 2 study of the compound of formula (I) was performed to determine its efficacy in the treatment of solid tumors.

Patients with solid tumors who had exhausted standard treatment options received the compound of formula (I) as a 1 hr infusion twice-weekly for 3 weeks (wks) of a 28 day cycle until disease progression. Serial PK and pharmacodynamic samples were obtained during cycle 1. Safety assessments included frequency and grade of adverse events (AEs), laboratory parameters and ECG changes.

Data were presented for 49 patients (22 M, 27 F; median age 55 yrs, range 32-81; ECOG status range 0-2) treated at doses from 2-144 mg/m². Patients received a median of 2 (range 1-12) cycles of the compound of formula (I). AEs reported in ≧20% of patients treated at doses from 2-120 mg/m² were fatigue, diarrhea, nausea, anemia, abdominal pain, constipation, anorexia, vomiting, and headache; the majority of events were mild to moderate in severity with absence of severe liver, ocular, cardiac and renal toxicity. Two DLTs (elevated transaminases) were reported in the 10 and 144 mg/m² cohorts. The compound of formula (I) showed linear PK, rapid distribution, a mean terminal half-life of 10-14 hours, a volume of distribution greater than total body water and no accumulation in plasma. A confirmed durable PR by RECIST was seen in a patient with metastatic melanoma. Additionally, 2 NSCLC patients who received 6 months of treatment had durable SD, with tumor shrinkage.

In summary, the compound of formula (I) was well-tolerated administered twice-weekly. Preliminary safety profile, activity signals and differences in client protein kinetics warrant continued evaluation of the compound of formula (I) using a twice-weekly dosing regimen.

### Example 6

### A Phase 2 Trial of the Compound of Formula (I): Efficacy and Safety in Patients with Metastatic Breast Cancer (MBC)

A phase 2 trial was performed to determine the safety and efficacy of the compound of formula (I) in the treatment of subjects with metastatic breast cancer.

Patients with locally advanced or MBC were treated with single agent of the compound of formula (I) at 200 mg/m² on a cycle of once weekly for 3 weeks, one week off, on a 28 day cycle. The primary endpoint of the trial was overall response rate using RECIST 1.1. Patients with HER2+ breast cancer were required to have received prior therapy with trastuzumab. No more than 3 lines of chemotherapy in the metastatic setting were permitted, but there was no limit on prior lines of hormone therapy. Patients were evaluated for response after 2 cycles. The trial used a Simon two-stage design requiring at least 3 responses among the first 22 patients, to allow expansion to a total of 40 patients.

A total of 22 patients were treated with a median age of 51 years (38 to 70) and the following subtypes: 13 HER2+ (10 ER+/HER2+; 3 ER−/HER2+), 6 ER+/HER2−, and 3 ER−/PR−/HER2−(TNBC).

Prior treatment regimens are summarized as follows:

| | Number of Subjects |
|---|---|
| Prior lines of | |
| chemotherapy in | |
| metastatic setting | |
| 1 | 8 |
| 2 | 9 |
| 3 | 5 |
| Prior lines of | |
| trastuzumab in | |
| metastatic setting | |
| 0 | 9 |
| 1 | 6 |
| 2 | 6 |
| 3 | 1 |

The responses of the subjects by ER, PR, and HER2 status are summarized in the table below.

| | | ER+/HER2− | HER2+ | TNBC |
|---|---|---|---|---|
| Response | Total (N = 22)* | (N = 6) | (N = 13)* | (N = 3) |
| ORR | 2 (9%) | 0 | 2 (9%) | 0 |
| CR | 0 | 0 | 0 | 0 |
| PR | 2 (9%) | 0 | 2 | 0 |
| SD | 7 (32%) | 0 | 6 (27%) | 1 (5%) |
| CBR* | 2 (9%) | 0 | 2 (9%) | 0 |

*(CR + PR + SD > 6 months)

These were the first data showing an objective anti-tumor response with single agent Hsp90 inhibitor therapy in patients with advanced breast cancer. Additionally, these were the first data to show anti-tumor activity for an Hsp90 inhibitor in TNBC. In this study, the single agent of the compound of formula (I) was well tolerated, with expected GI toxicity that was mild in nature and manageable in all patients.

### Example 7

### The Compound of Formula (I) Displays Activity Across Breast Cancer Subtypes

Breast cancer is a heterogeneous disease historically broken down into 4 subtypes. Various compounds were tested for their effects in cell viability assays using various breast cancer cell lines. Cellular viability was assessed using the CellTiter-Glo® Luminescent Cell Viability Assay (Promega, Madison, Wis., USA) according to the manufacturer's protocol. KRAS mutant NSCLC cell lines were seeded into 96-well plates based on optimal growth rates determined empirically for each line. Twenty-four hours after plating, cells were dosed with graded concentrations of the compound of formula (I) for 72 h. CellTiter-Glo® was added (50% v/v) to the cells, and the plates incubated for 10 min prior to luminescent detection in a SpectraMax® Plus 384 microplate reader (Molecular Devices, Sunnyvale, Calif., USA). Data were normalized to percent of control and IC₅₀ values used to determine the sensitivity of each line. For the comparative analysis study with MEK and PI3K/mTOR inhibitors, A549, H2009, Calu-1, and H358 cells were treated with graded concentrations of the compound of formula (I), AZD6244, or BEZ235 for 72 h and cell viability measured as above.

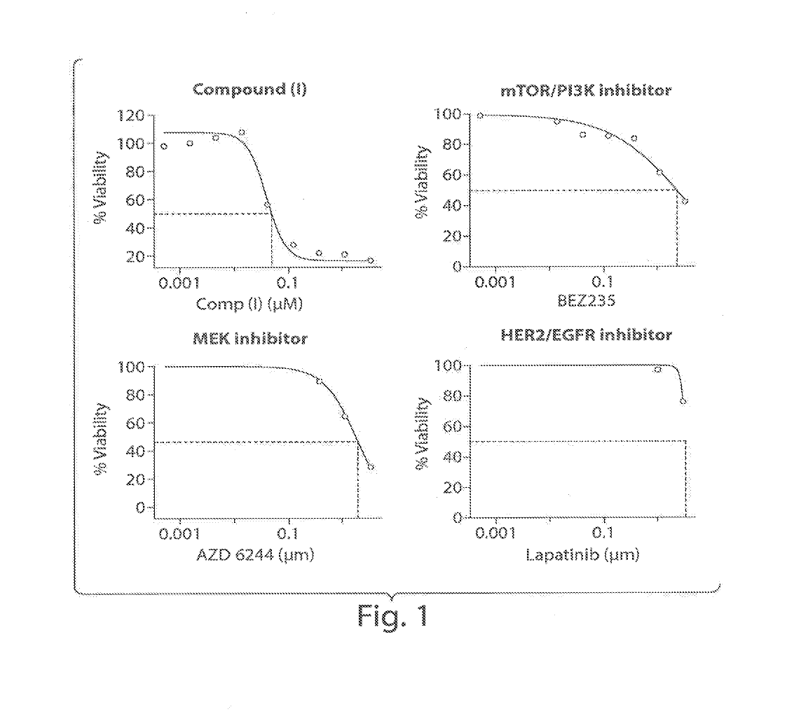

Shown in FIG. 1, the compound of formula (I) showed potency across all 4 subtypes (luminal HER2+, luminal HER2−, Basal A, Basal B) of breast cancer cells, grown as a monolayer in vitro. The IC50s of the various compounds and the ER, PR, and HER2 status are provided in the table below.

Breast cancer, cell lines, marker status, and IC50 in response to the compound of formula (I) at 72 hr (viability by CTG).

| Cell Line | Subtype | ER | PR | HER2 | IC50, nM |
|---|---|---|---|---|---|
| OCUB-M | Basal | — | — | | 39 |
| MDA-MB-468 | Basal A | — | — | | 27 |
| HCC70 | Basal A | — | — | | 114 |
| MDA-MB-231 | Basal B | — | — | | 24 |
| SK-BR-3 | Luminal | — | — | + | 10 |
| BT-474 | Luminal | + | + | + | 13 |
| MCF7 | Luminal | + | + | | 25 |

Basal breast cancer is a subtype believed to be more stem like and less differentiated than luminal breast cancer, and therefore more aggressive with limited treatment options. Comparison was made for the anticancer activity of the compound of formula (I) versus MEK and mTOR inhibitors in the basal line MDA-MB-231, using lapatinib as a control since these cells were HER2 negative. Shown in FIG. 1, the compound of formula (I) was highly potent, killing all the cells as opposed to the weak activity of the mTOR and MEK inhibitors.

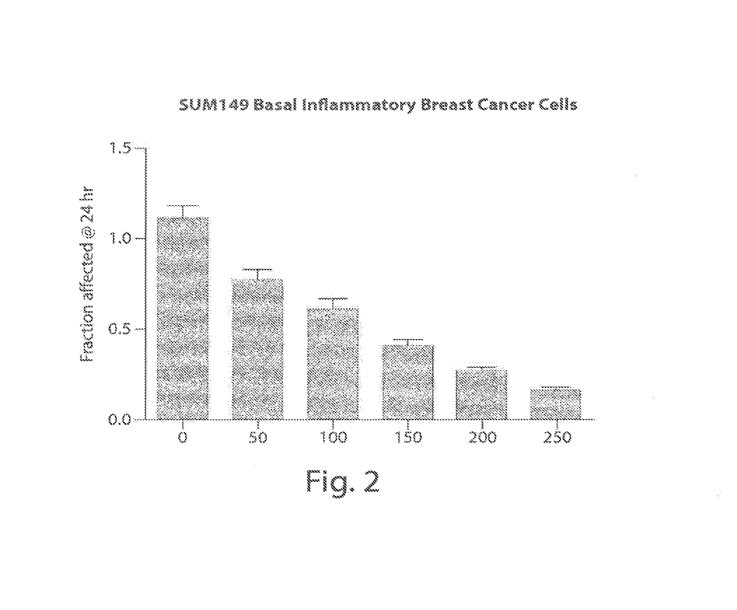

The compound of formula (I) was assayed in inflammatory breast cancer (IBC), a rare but aggressive form of breast cancer distinct from the subtypes presented above. Shown in FIG. 2, the compound of formula (I) displayed considerable anticancer activity against SUM149 cells 24 hr after exposure.

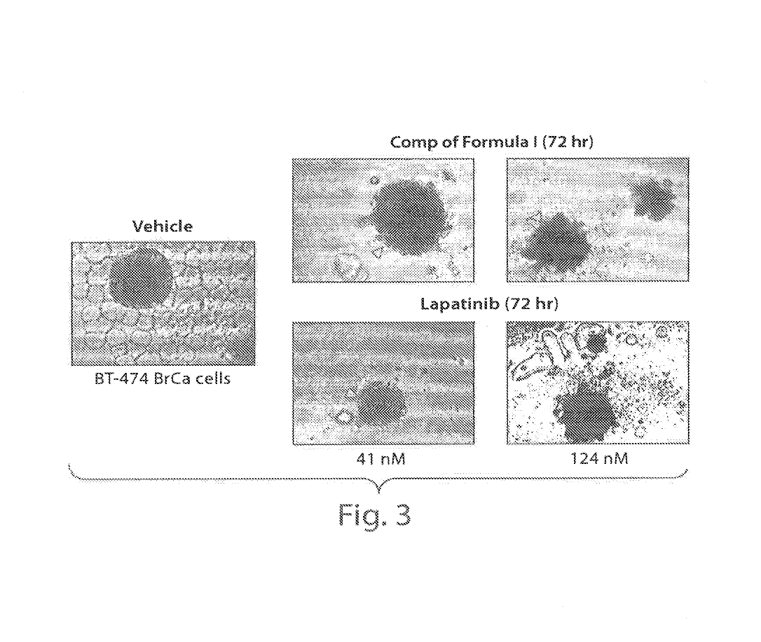

BT-474 HER2+ luminal cells were cultured as mammospheres in Matrigel® and exposed to the compound of formula (I) for 72 hr. As shown in FIG. 3, the compound of formula (I) was fully capable of killing cells organized into spheroids, with an IC50 (20 nM) nearly identical to that observed in 2D (13 nM), demonstrating that the compound of formula (I) retained its activity in breast cancer cells grown in three dimensions.

### Example 8

### Expression of HSP90 Client Proteins in BT-474 HER2+Luminal Breast Cancer Cells after Treatment with the Compound of Formula (I)

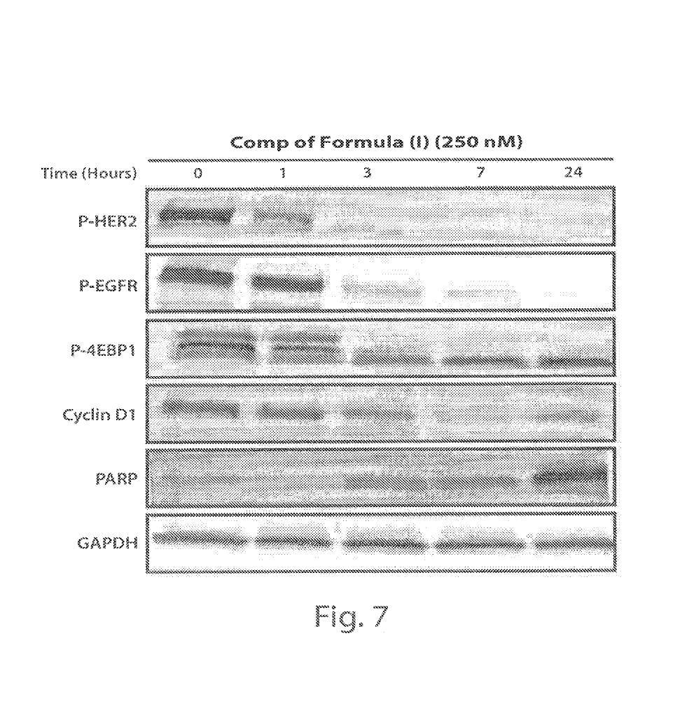

Expression of various proteins in the BT-474 HER2+ luminal breast cancer cells after exposure to the compound of formula (I) was assessed by western blot using routine methods. Briefly, following treatment, tumor cells were disrupted in lysis buffer (CST) on ice for 10 min. Lysates were clarified by centrifugation and equal amounts of proteins resolved by SDS-PAGE before transfer to nitrocellulose membranes (Invitrogen, Carlsbad Calif.). Membranes were blocked with 5% skim milk in TBS with 0.5% Tween and immunoblotted with the indicated antibodies. Antibody-antigen complexes were visualized using an Odyssey system (LI-COR, Lincoln, Nebr.). FIG. 7 is a western blot showing expression of various proteins in the BT-474 HER2+ cells at various time points after treatment with the compound of formula (I).

### Example 9

### Treatment of breast cancer with the compound of formula (I) and BEZ235 in a mouse xenograft tumor model

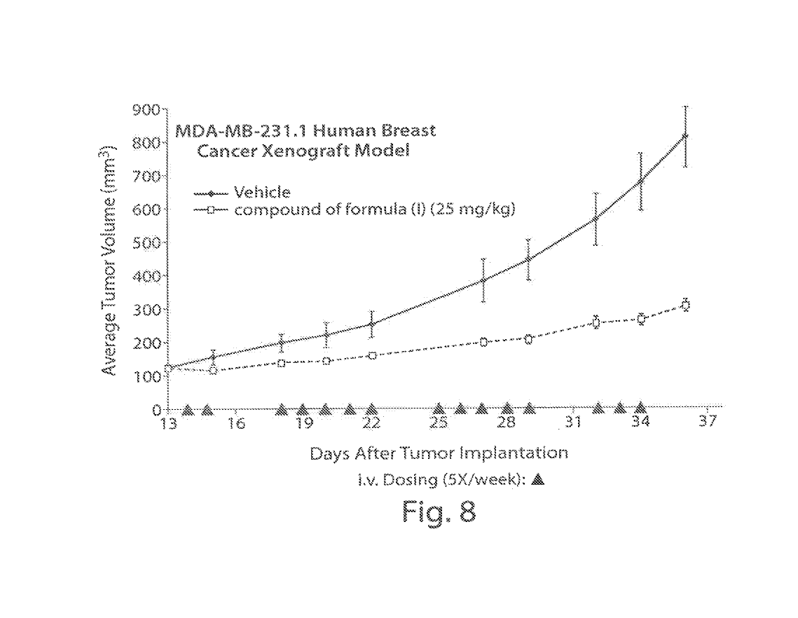

Female immunodeficient CD-1 (nude) mice (Charles River Laboratories, Wilmington, Mass.) were maintained in a pathogen-free environment, and all in vivo procedures were approved by the Synta Pharmaceuticals Corp. Institutional Animal Care and Use Committee. A549 NSCLC cells (7.5×10⁶) were subcutaneously implanted into the animals. Mice bearing established tumors (100-200 mm³) were randomized into treatment groups of 8 and i.v. dosed via the tail vein with either vehicle, the compound of formula (I) formulated in 10/18 DRD (10% DMSO, 18% Cremophor RH 40, 3.6% dextrose, 68.4% water) or p.o. dosed with BEZ235 formulated in PEG300/NMP (90% PEG300, 10% N-Methylpyrrolidone) Animals were treated with the compound of formula (I) at 50 mg/kg weekly or BEZ235 at 10 mg/kg 5 times a week, either alone or in combination. Tumor growth inhibition was determined as described previously. See Proia et al, PLoS One. 2011; 6(4):e18552. The results are shown in FIG. 8.

As shown in FIG. 8, average tumor volume was significantly reduced in mice treated with the compound of formula (I) and BEZ235 as compared to vehicle, particularly at later time points. The efficacy of the compound of formula (I) and BEZ235 were about the same, and average tumor volume substantially reduced in mice treated with compound of formula (I) and BEZ235 as compared to vehicle control, particularly at later time points.

In summary, the compound of formula (I) displayed anticancer activity in all four breast cancer subtypes, as well as inflammatory breast cancer Importantly, the compound of formula (I) was equally effective in killing cells grown as three dimensional spheres compared to cells grown in monolayer, as well as in vivo.

### Example 10

### Compound of Formula (I) Displays Activity Across GIST Subtypes

Many of the oncoproteins associated with gastric cancer and Hsp90 client proteins including HER2, MET, RAS and the FGFR family. The compound of formula (I) was evaluated for its effect on the growth of AGS (wt-p53 and mut-KRAS) and MKN45 (wt-p53, wt-KRAS, MET amplified) gastric cancer cells. Cells were treated for 72 hr and viability determined by CTG (upper) or Syto60 (lower). Most gastric cell lines displayed low nanomolar IC₅₀ with the compound of formula (I), as shown in the Table below.

| | AGS | MKN45 |
|---|---|---|
| IC50 (nM) | wt-p53, mut-KRAS | wt-p53, wt-KRAS |
| Ganetespib | 1.5 | 0.5 |
| Docetaxel | 0.3 | 0.1 |
| 17-AAG | 50 | 1.0 |

| Cell Line | Comp of formula (I) IC50 (nM) |
|---|---|
| AGS | 5 |
| SNU-1 | 6 |
| GT3TKB | 6 |
| MKN-45 | 8 |
| GCIY | 9 |
| HGC-27 | 11 |
| ECC12 | 12 |
| SNU-5 | 31 |
| Hs746T | 384 |

The compound of formula (I) was also evaluated for its affects on Hsp90 client proteins in AGS gastric cancer cells by western blot. The compound of formula (I) abolished the expression of EGFR, IGF-IR, C-RAF and their down-stream effectors PI3K/AKT and MAPK, resulting in PARP cleavage and increased levels of p-Histone H2X (Ser139), a marker for DNA fragmentation during apoptosis. Similar to the observation in melanoma cells, exposure to the compound of formula (I) enhanced B-RAF expression. Without being bound by mechanism, it is suggested that a decrease in phosphorylation of CDK1 by the compound of formula (I) may be due to the loss of WEE1 expression, an important regulatory kinase for CDK1. In MKN45 cells, exposure of the compound of formula (I) led to the complete degradation of MET and IGF-IR, followed by inactivation of AKT.

In summary, the compound of formula (I) displayed potent anticancer activity with low nanomolar IC50s in gastric cancer cell lines. Without being bound by mechanism, it is suggested that the activity is at least, in part, a result of widespread degradation of client proteins essential for cell growth, proliferation and survival including MET, IGF-1R, EGFR, WEE1 and CDK1.

### Example 11

### Compound of Formula (I) Displays Efficacy in Head and Neck Cancer Subtypes

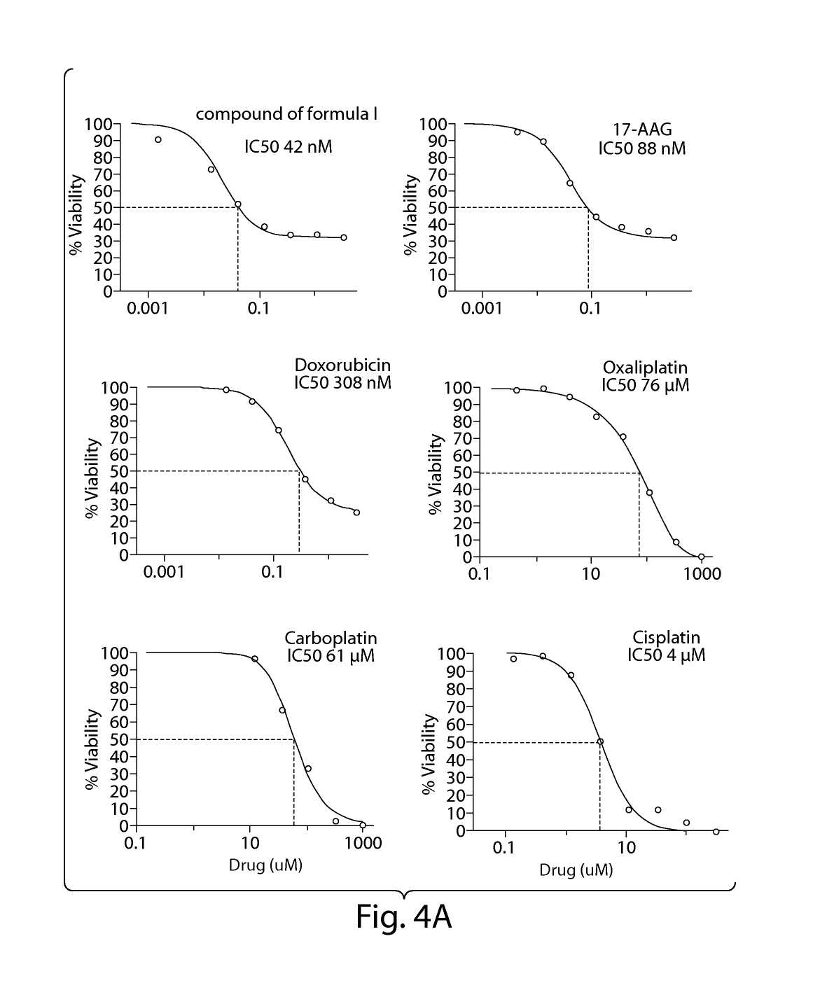

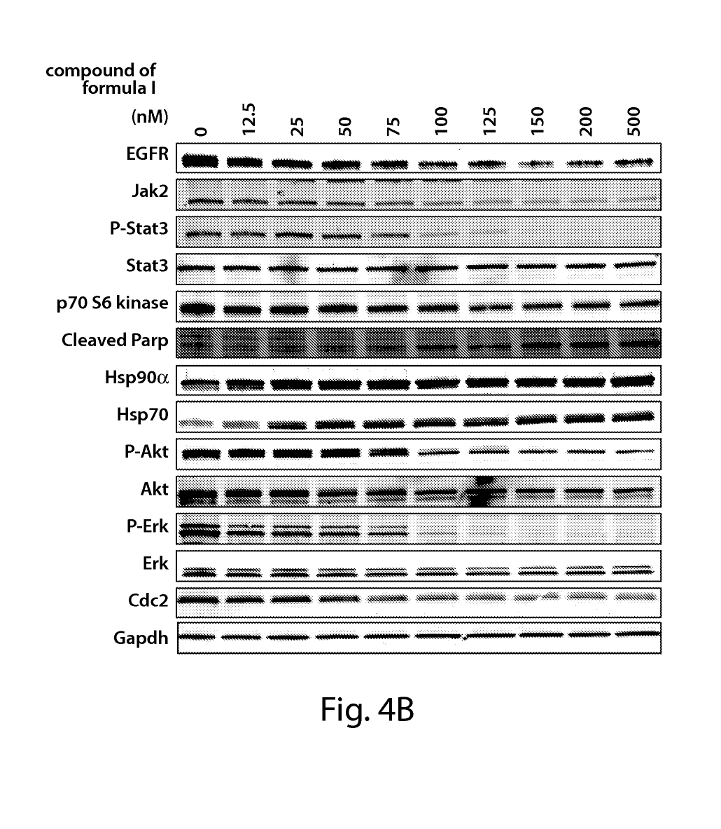

Head and neck (H/N) cancer refers to a group of biologically similar cancers originating from the upper autodigestive tract. First line therapies include EGFR inhibitors and platins. Modulation of EGFR and other client proteins by the compound of formula (I) was investigated in Detroit 562 H/N cancer cells. As shown in FIG. 4B, the compound of formula (I) led to the depletion of EGFR and JAK2, resulting in the inactivation of several key effectors including AKT, STAT3, p70S6, and ERK followed by cleaved PARP. Single agent viability analyses were then performed and it was found that the IC₅₀ of the compound of formula (I) (42 nM) correlated with initiation of client protein degradation (FIG. 4A). A fraction of cells remained viable after a 72 hr exposure to the compound of formula (I), in contrast to the platins which completely killed the cells.

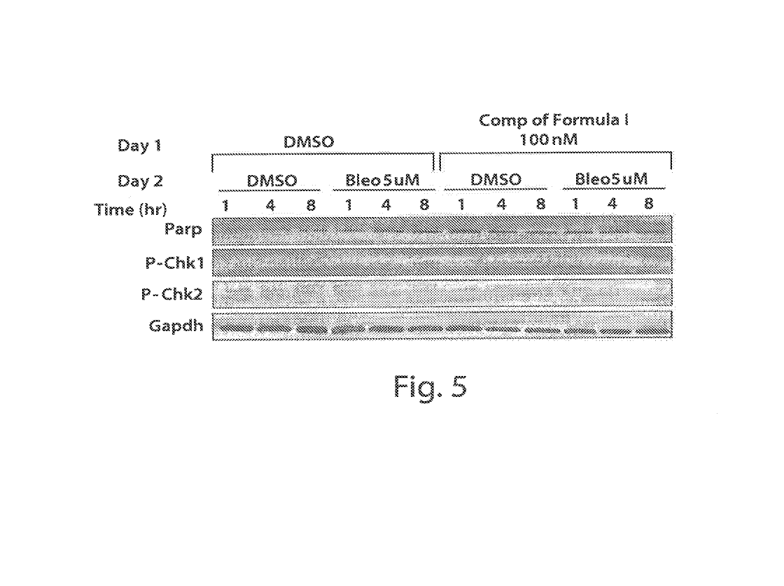

Western blot of protein expression is shown in FIG. 5. Cell extracts from Detroit 562 head and neck cancer cells were treated with 100 nM of the compound of formula (I) 24 hours prior to receiving the DNA damaging agent bleomycin (5 μM). Protein expression was measured at the indicated time points after bleomycin treatment. Bleomycin increased both Chk1 and Chk2 phosphorylation, which was blocked when cells were treated first with compound of formula (I).

### Example 12

### Compound of Formula (I) in Combination with Standard of Care Chemotherapies Displays Efficacy in NSCLC Cancer Subtypes with KRAS Mutations

Mutant KRAS is detected in 20-25% of non-small cell lung carcinomas (NSCLC) and represents one of the most common oncogenic drivers of this disease. NSCLC tumors with oncogenic KRAS respond poorly to currently available therapies necessitating the pursuit of new treatment strategies. Recent results from a Phase 2 trial with the compound of formula (I) revealed that >60% of patients with NSCLC having a KRAS mutation exhibited tumor shrinkage at 8 weeks, indicating that the compound of formula (I) is useful in the treatment of this disease.

To further understand the actions of the compound of formula (I) in NSCLC tumors having a KRAS mutation, studies were executed in a diverse panel of KRAS mutant NSCLC cell lines to investigate whether the compound of formula (I) is effective in suppressing critical cell signaling nodes responsible for KRAS-driven NSCLC cell survival and to assess whether the compound of formula (I) can synergize with both clinical agents targeted against these signaling nodes and standard of care chemotherapies.

For combinatorial analysis, cells were seeded in 96-well plates at a predetermined, optimum growth density for 24 h prior to the addition of drug or vehicle to the culture medium. Drug combinations were applied at a non-constant ratio over a range of concentrations for 72 or 96 hours. For each compound tested, a 7 point dose range was generated based on 1.5 fold serial dilutions using IC₅₀ values set as the mid-point. Cell viability was assessed by either AlamarBlue® (Invitrogen, Carlsbad, Calif.) or CellTiter-Glo® assays and normalized to vehicle controls. For each combination study, the level of growth inhibition (fraction affected) is plotted relative to vehicle control. Data are presented as one relevant combination point and the corresponding single agent data for each cell line tested.

The compound of formula (I) displayed potent anticancer activity across 15 KRAS mutant NSCLC cell lines assayed in vitro, with an average IC₅₀ of 24 nM. Combining the compound of formula (I) with anti-mitotics, alkylating agents or topoisomerase inhibitors resulted in an increase in cell death of up to 44, 61 and 26%, respectively, versus monotherapy. At the molecular level, the compound of formula (I) induced the destabilization of several KRAS substrates, including BRAF and CRAF, leading to inactivation of their downstream effectors followed by programmed cell death. The compound of formula (I) effectively suppressed the growth of human KRAS mutant NSCLC tumor xenografts in vivo; however, the compound of formula (I) did not induce tumor regression. In light of this, we sought to investigate whether inhibitors targeting KRAS driven signaling nodes would confer greater sensitivity to the compound of formula (I). In vitro, combinations of low dose of the compound of formula (I) with either MEK or PI3K/mTOR inhibitors consistently resulted in greater activity than monotherapy, up to 77% and 42%, respectively. Furthermore, the compound of formula (I) suppressed activating feedback loops that occur in response to MEK and PI3K/mTOR inhibition, providing a rationale for the enhanced combinatorial activity. To validate these results, in vivo combinations were performed with the compound of formula (I) and a PI3K/mTOR inhibitor in KRAS mutant NSCLC xenografts. While both agents promoted tumor shrinkage on their own, considerable improvement in tumor growth inhibition was observed in the combination arm.

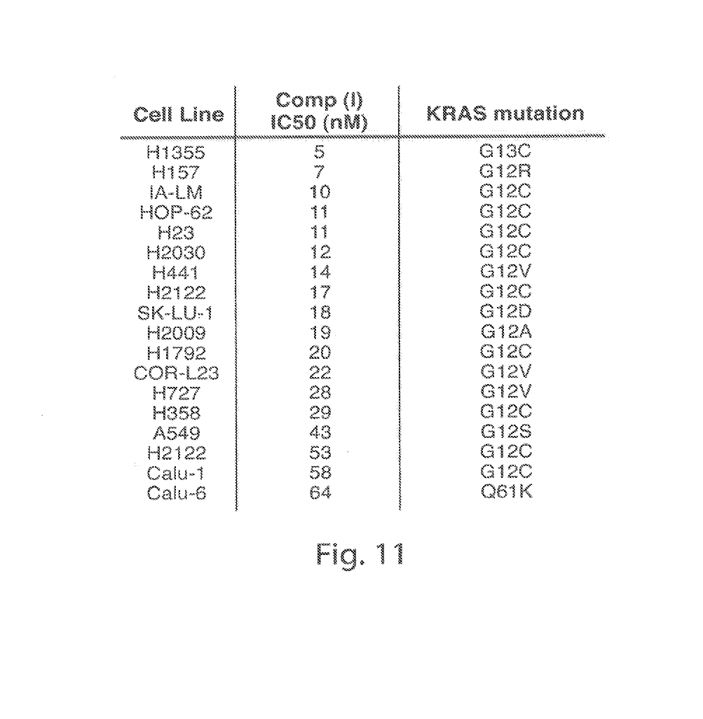

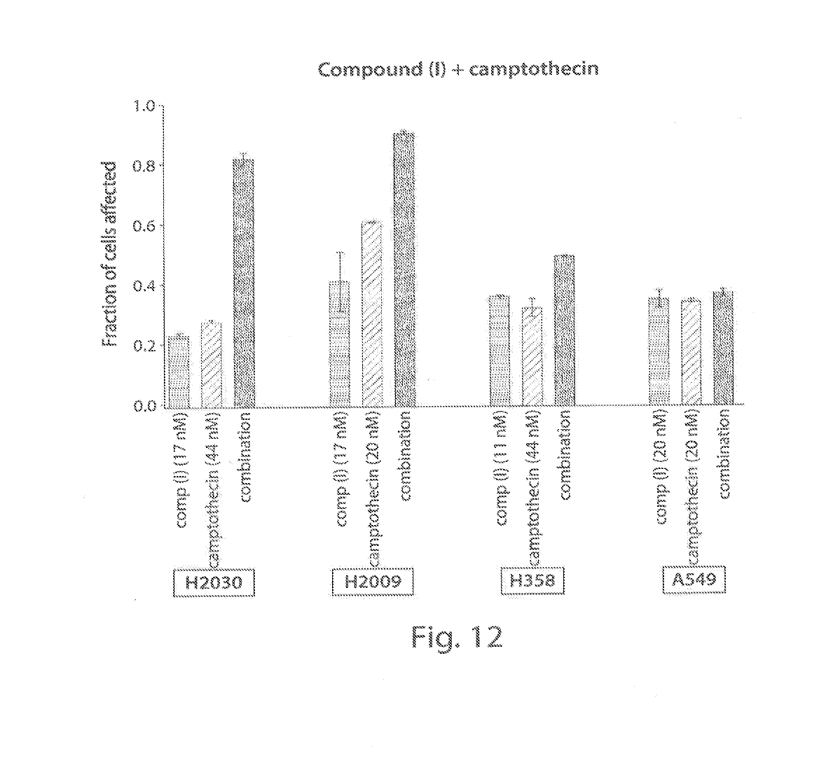

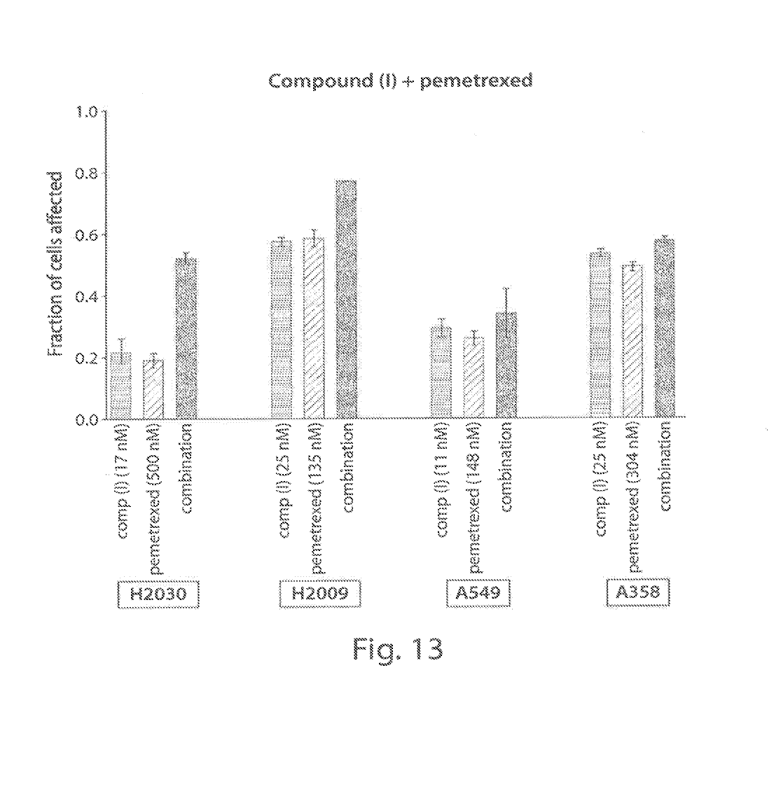

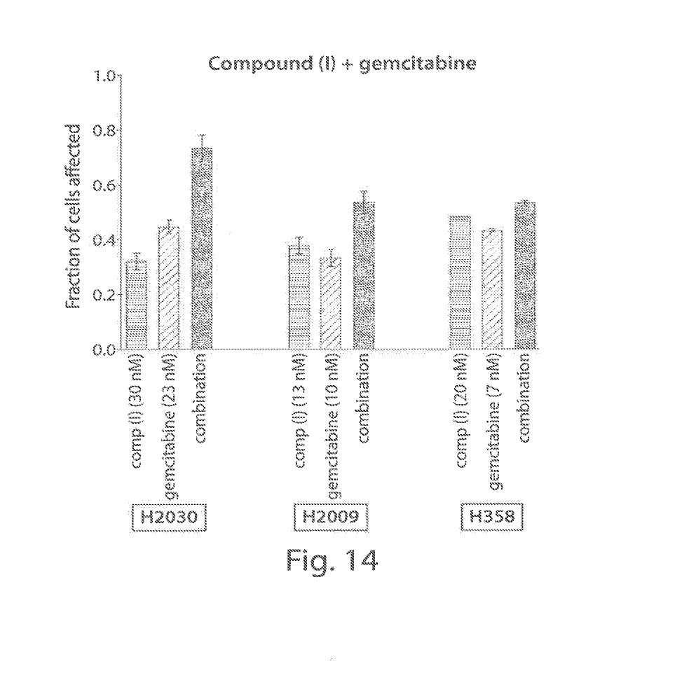

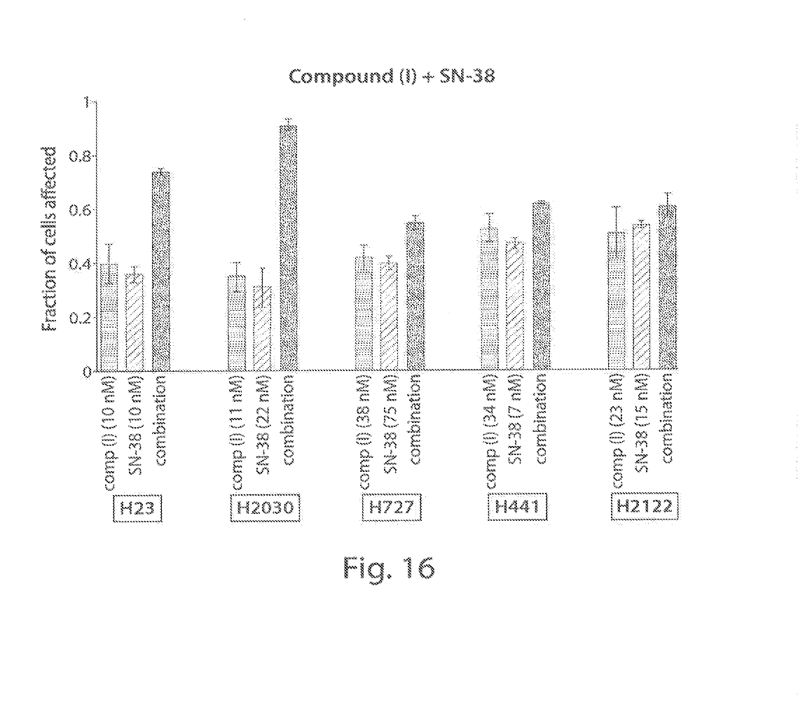

More particularly, the compound of formula (I) elicited promising activity against mutant KRAS NSCLC tumor cells (FIG. 11). In order to further identify feasible strategies to enhance the anti-tumor activity of the compound of formula (I), combination studies were performed with standard of care chemotherapies in mutant KRAS NSCLC cell lines. Combining low nanomolar concentrations of the compound of formula (I) with the topoisomerase I inhibitor camptothecin resulted in a 1.5, 3.4, and 1.4 fold increase in cytotoxicity for H2009, H2030, and H358 cells, respectively (FIG. 12). Similar results were observed for SN-38, another topoisomerase I inhibitor (FIG. 16). It was also found that combining the compound of formula (I) with the antimetabolite pemetrexed enhanced cell death by 2.4 and 1.5 fold for H2030 and H2009 cells, respectively, while a marginal increase in cytotoxicity was observed for A549 and H358 cells (FIG. 13). The compound of formula (I) in combination with the nucleoside analog, gemcitabine, increased cell death 2.3 and 1.4 fold for H2009 and A549 cells, respectively, and no benefit was observed for H358 cells (FIG. 14).

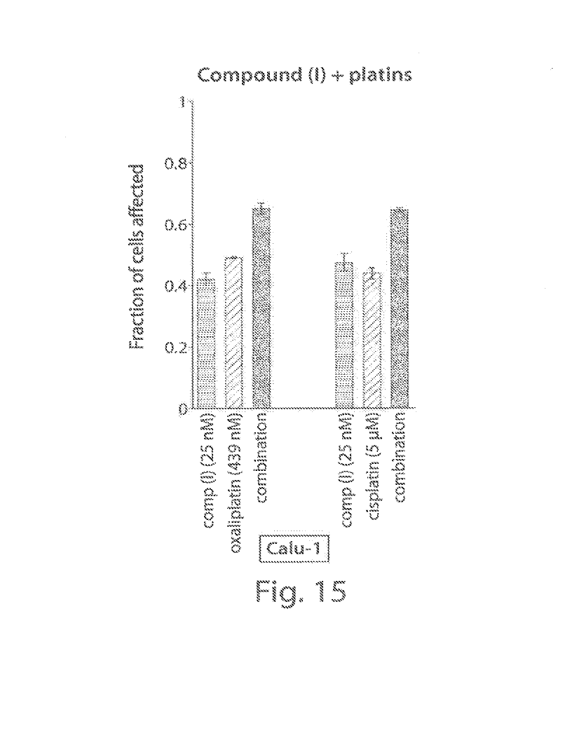

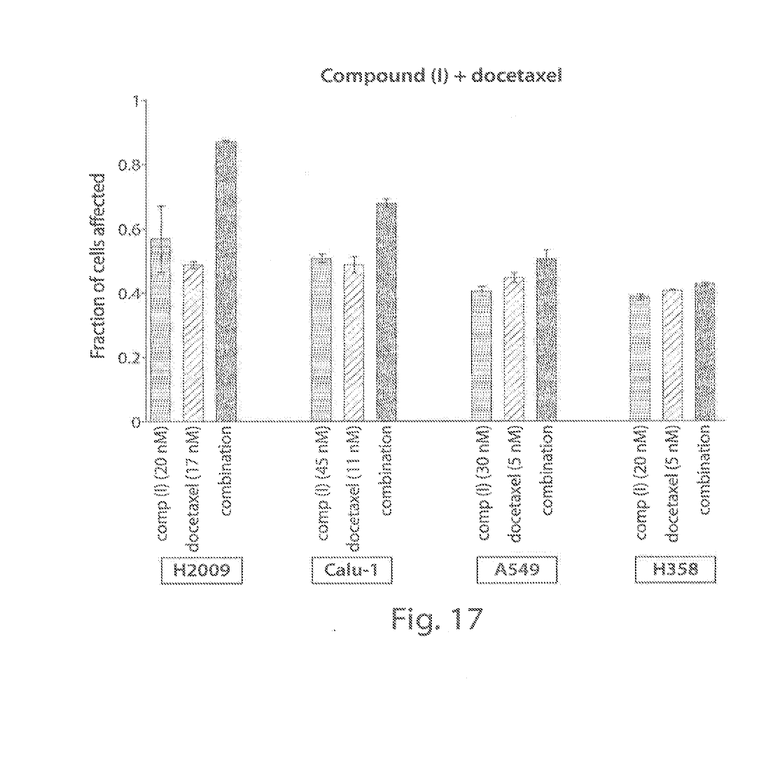

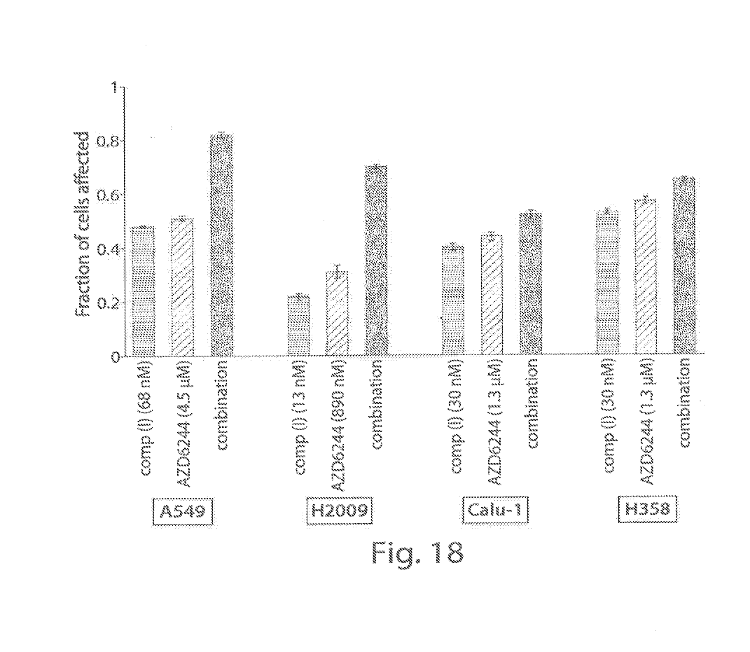

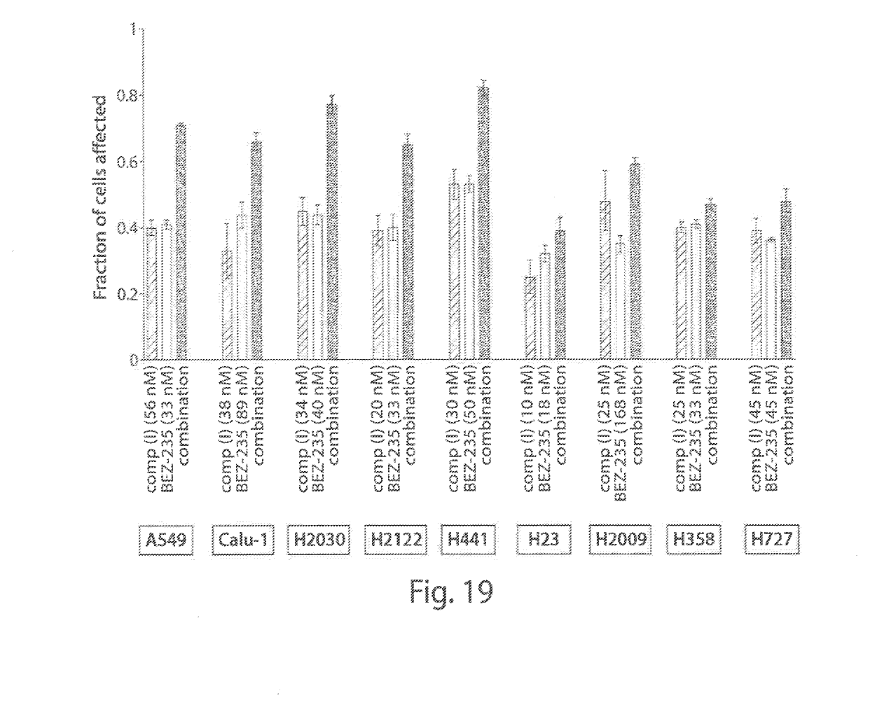

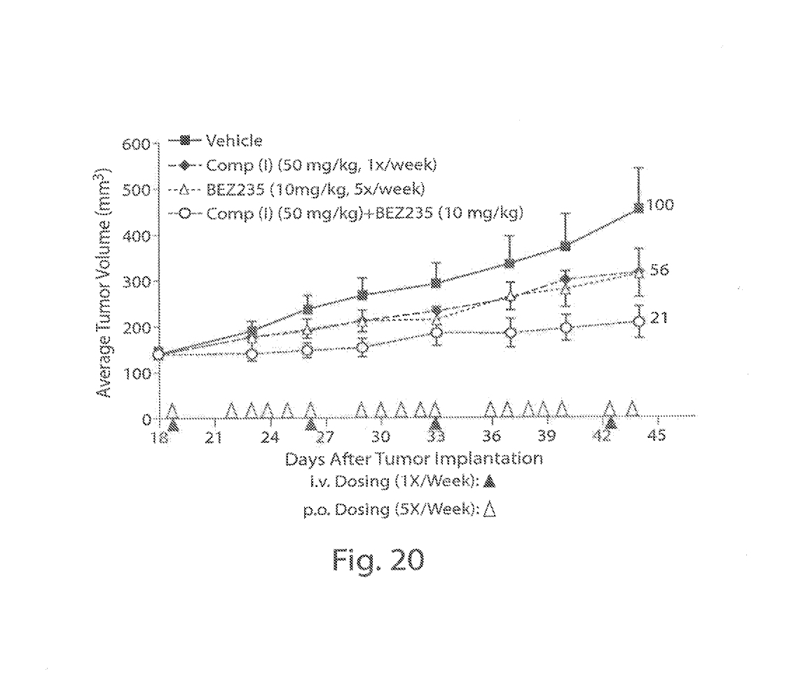

More combination data are presented in FIGS. 15-20. The results highlight the heterogeneity in response to various targeted agents and chemotherapies as well as the variability in benefit achieved when these agents are combined with the compound of formula (I). Taken together, these results suggest that chemotherapies currently used for the treatment of NSCLC may enhance the antitumor activity of the compound of formula (I).

Without being bound by mechanism, it is suggested that the compound of formula (I) promotes destabilization of multiple oncogenic signaling proteins and is potently cytotoxic in KRAS mutant NSCLC cells and simultaneously disrupts multiple nodes of KRAS driven signaling resulting in enhanced apoptosis compared to MEK or PI3K/mTOR inhibitors. Combining the compound of formula (I) with MEK or mTOR inhibitors blocks feedback induced accumulation of activated MEK and ERK contributing to enhanced cytotoxicity in vitro and in vivo. Common standard of care chemotherapeutics utilized in the treatment of NSCLC enhance the activity of the compound of formula (I).

In summary, the compound of formula (I), a potent inhibitor of Hsp90, has shown encouraging evidence of clinical activity, including tumor shrinkage in patients with KRAS mutant NSCLC. In vitro, the compound of formula (I) exhibited potent anticancer activity in NSCLC cells with a diverse spectrum of KRAS mutations due in part to degradation and inactivation of critical KRAS signaling effectors. Combination with targeted therapies that overlap with these signaling nodes led to enhanced anticancer activity in vitro and in mouse models of KRAS mutant NSCLC. Taken together, these results demonstrate clinical utility of the compound of formula (I) in patients with KRAS mutant NSCLC.

Standard of care chemotherapeutics utilized in KRAS mutant NSCLC show activity with the compound of formula (I) in vitro. Camptothecin, pemetrexed and gemcitabine showed up to 4 fold increases in cell death when combined with the compound of formula (I). None of the agents antagonized the anticancer activity of the compound of formula (I).

### Example 13

### Phase 1 trial of the combination of the compound of formula (I) and docetaxel in the treatment of solid tumors

A Phase 1 study of the compound of formula (I) in combination with docetaxel in solid tumors has been studied in a broad range of clinical trials.

A trial to evaluate three dose-level combinations of docetaxel and the compound of formula (I), administered on a three-week cycle, with the primary objective of determining an optimal dose for future clinical trials was performed. Docetaxel was administered as a one hour IV infusion on day 1 and the compound of formula (I) was administered as a one hour IV infusion on days 1 and 15. The dose level combinations evaluated were 150 mg/m² and 60 mg/m²; 150 mg/m² and 75 mg/m²; and 200 mg/m² and 75 mg/m² for the compound of formula (I) and docetaxel respectively. The standard of care dose level for docetaxel was 75 mg/m². A total of 19 patients received at least one dose of study treatment at the cut-off time. The median number of cycles of treatment was 4, with a range of 1 to 11 cycles of treatment. No prophylactic treatment for neutropenia was used. The combination of the compound of formula (I) at 150 mg/m² and docetaxel at 75 mg/m² was selected as the recommended dose.

It was observed that a patient responded with over 50% shrinkage of target tumor lesions on the trial diagnosed with cancer of the parotid gland, the largest of the salivary glands. The patient did not respond to prior treatment regimens including carboplatin, cetuximab, and methotrexate.

The most common adverse event was neutropenia (67%), including four patients (22%) who reported febrile neutropenia. Neutropenia, a known effect of docetaxel treatment, was commonly observed at approximately 8 days following dosing and typically resolved spontaneously within 7 days. Serious adverse events were reported in a total of nine patients (50%) including two reports of pneumonia and one report each of chest pain, chills, dyspnea, fatigue, mucosal inflammation, neutropenia, pneumothorax, pulmonary embolism, rib fracture, transient ischaemic attack, and vomiting.

Pharmacokinetic data indicate a pharmacokinetic similarity between the compound of formula (I) administered alone and the compound of formula (I) administered prior to docetaxel. There was no effect of the compound of formula (I) on docetaxel pharmacokinetics.

These results support the use of the compound of formula (I) at a dose of 150 mg/m² in combination with docetaxel at a dose of 75 mg/m² for treating NSCLC and other solid cancers.

All publications cited herein are incorporated by reference in their entirety. In case of conflict, the present specification, including definitions, will control. In addition, the materials, methods, and examples throughout the specification are illustrative only and not intended to be limiting in any way.

What is claimed is:

1. A method of treating cancer in a subject, comprising administering a compound of formula (I) or a pharmaceutically acceptable salt or tautomer thereof: 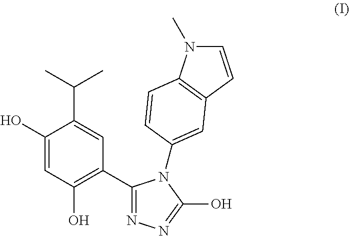 [SMILES: CC(C)c1cc(-c2nnc(O)n2-c2ccc3c(ccn3C)c2)c(O)cc1O]
wherein the subject has a cancer with a mutation in KRAS.

2. The method of claim 1, wherein the cancer is selected from the group consisting of lung cancer, breast cancer, gastric cancer, colorectal cancer, pancreatic cancer, ocular melanoma, prostate cancer, gastrointestinal stromal tumors (GIST), advanced esophagogastric cancer, melanoma, hepatocellular cancer, solid tumor, liver cancer, head and neck cancer, small cell lung cancer, and non-small cell lung cancer (NSCLC).

3. The method of claim 1 or 2, wherein the cancer is NSCLC.

4. The method of claim 3, wherein the NSCLC was previously treated and not responsive.

5. The method of claim 3 or 4, wherein the NSCLC was previously treated with crizotinib and is no longer responsive to the crizotinib treatment.

6. The method of claim 1 or 2, wherein the cancer is breast cancer.

7. The method of claim 6, wherein the breast cancer is HER2 positive, and has been previously treated with trastuzumab.

8. The method of claim 6 or 7, wherein the breast cancer is HER2 positive and trastuzumab refractory.

9. The method of claim 6, wherein the cancer is triple negative breast cancer.

10. The method of claim 2, wherein the prostate cancer is metastatic hormone-resistant prostate cancer, or metastatic castration-resistant prostate cancer.

11. The method of any one of claims 1 to 10, further comprising identifying a subject as having a cancer with a mutation in KRAS.

12. A method of treating cancer in a subject, comprising administering a compound of formula (I) or a pharmaceutically acceptable salt or tautomer thereof: 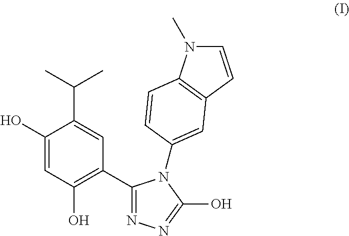 [SMILES: CC(C)c1cc(-c2nnc(O)n2-c2ccc3c(ccn3C)c2)c(O)cc1O]
wherein the subject has a cancer with an ALK mutation.

13. The method of claim 12, wherein the cancer is selected from the group consisting of lung cancer, breast cancer, gastric cancer, colorectal cancer, pancreatic cancer, ocular melanoma, prostate cancer, gastrointestinal stromal tumors (GIST), advanced esophagogastric cancer, melanoma, hepatocellular cancer, solid tumor, liver cancer, head and neck cancer, small cell lung cancer, and non-small cell lung cancer (NSCLC).

14. The method of claim 12 or 13, wherein the cancer is NSCLC.

15. The method of claim 14, wherein the NSCLC was previously treated and not responsive.

16. The method of claim 14 or 15, wherein the NS CLC was previously treated with crizotinib and is no longer responsive to the crizotinib treatment.

17. The method of claim 12 or 13, wherein the cancer is breast cancer.

18. The method of claim 17, wherein the breast cancer is HER2 positive, and has been previously treated with trastuzumab.

19. The method of claim 17 or 18, wherein the breast cancer is HER2 positive and trastuzumab refractory.

20. The method of claim 17, wherein the cancer is triple negative breast cancer.

21. The method of claim 13, wherein the prostate cancer is metastatic hormone-resistant prostate cancer, or metastatic castration-resistant prostate cancer.

22. The method of any one of claims 12 to 21, further comprising identifying a subject as having a cancer with a mutation in ALK.

23. A method of treating breast cancer in a subject, comprising administering a compound of formula (I) or a pharmaceutically acceptable salt or tautomer thereof: 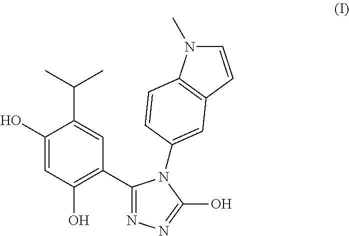 [SMILES: CC(C)c1cc(-c2nnc(O)n2-c2ccc3c(ccn3C)c2)c(O)cc1O]
wherein the breast cancer is selected from the group consisting of metastatic breast cancer, triple negative breast cancer, and HER2 positive breast cancer.

24. The method of claim 23, wherein the breast cancer is HER2 positive breast cancer, HER2 negative breast cancer, Basal A breast cancer, Basal B breast cancer, and inflammatory breast cancer.

25. The method of claim 23 or 24, wherein the breast cancer is HER2 positive, and has been previously treated with trastuzumab.

26. The method of any of claims 23 to 25 wherein the breast cancer is HER2 positive and trastuzumab refractory.

27. The method of any of claims 23 to 26, further comprising identifying the subject as having a metastatic breast cancer.

28. The method of any of claims 23 to 26, further comprising identifying the subject as having a HER2+ breast cancer.

29. The method of any of claims 23 to 26, further comprising identifying the subject as having a triple negative breast cancer.

30. The method of any of claims 1-29, wherein the compound of formula (I) or a pharmaceutically acceptable salt or tautomer thereof, is administered at a dose of 2 mg/m² to 260 mg/m².

31. A method of treatment of cancer in a subject comprising administration of a compound of formula (I) or a pharmaceutically acceptable salt or tautomer thereof: 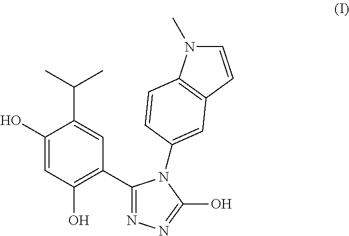 [SMILES: CC(C)c1cc(-c2nnc(O)n2-c2ccc3c(ccn3C)c2)c(O)cc1O]
at a dose of 2 mg/m² to 260 mg/m².

32. The method of any one of claims 1-31, wherein the method further comprises administering one or more additional anticancer agents.

33. The method of claim 32, wherein the one or more agents are selected from the group consisting of BEZ-235, AZD6244, AZD8055, SN-38, gemcitabine, camptothecin, docetaxel, cisplatin, oxaliplatin, crizotinib, paclitaxel, trastuzumab, and pemetrexed.

34. The method of any one of claims 1-33, wherein the amount of the compound of formula (I) administered is from 75 mg/m² to 260 mg/m².

35. The method of claim 34, wherein the amount of the compound of formula (I) administered is from 125 mg/m² to 260 mg/m².

36. The method of claim 35, wherein the amount of the compound of formula (I) administered is from 175 mg/m² to 260 mg/m².

37. The method of any one of claims 1-33, wherein the amount of the compound of formula administered is 75 mg/m², 85 mg/m², 100 mg/m², 110 mg/m², 115 mg/m², 120 mg/m², 145 mg/m², 150 mg/m², 175 mg/m², 180 mg/m², 200 mg/m², 215 mg/m² or 260 mg/m².

38. The method of any one of claims 1-37, wherein the compound of formula (I) is administered by intravenous infusion.

39. The method of claim 38, wherein the infusion is a peripheral intravenous infusion.

40. The method of claim 38, wherein the compound of formula (I) is infused over 60 minutes.
